SEQUENCE LISTING (28 sequences)

SEQ ID NO: 1 - protein, 27 aa, Conus purpurascens
  PEPTIDE (1)..(27): Xaa at residue 2, 7, 18, 19, 22 and 25 may be Arg, homoarginine, ornithine, Lys, N-methyl-Lys, N,N-dimethyl-Lys, N,N,N-trimethyl-Lys, any synthetic basic amino acid, His or halo-His; Xaa
CXIXNQXCXQXLDDCCSXXCNXXNXCV

SEQ ID NO: 2 - protein, 27 aa, Conus purpurascens
  PEPTIDE (1)..(27): Xaa is Hyp
CRIXNQKCFQHLDDCCSAKCNRFNKCV

SEQ ID NO: 3 - protein, 27 aa, Conus purpurascens
  PEPTIDE (1)..(27): Xaa is Hyp
CRIXNQKCFQHLDDCCSRKCNAFNKCV

SEQ ID NO: 4 - protein, 27 aa, Conus purpurascens
  PEPTIDE (1)..(27): Xaa is Hyp
CRAXNQKCFQHLDDCCSRKCNRFNKCV

SEQ ID NO: 5 - protein, 27 aa, Conus purpurascens
  PEPTIDE (1)..(27): Xaa is Hyp
CRIXNQKCFQHLDDCCSRACNRFNKCV

SEQ ID NO: 6 - protein, 27 aa, Conus purpurascens
  PEPTIDE (1)..(27): Xaa is Hyp
CAIXNQKCFQHLDDCCSRKCNRFNKCV

SEQ ID NO: 7 - protein, 27 aa, Conus purpurascens
  PEPTIDE (1)..(27): Xaa is Hyp
CRIXNQKCAQHLDDCCSRKCNRFNKCV

SEQ ID NO: 8 - protein, 27 aa, Conus purpurascens
  PEPTIDE (1)..(27): Xaa is Hyp
CRIXNQKCFQHLDDCCSRKCNRFNACV

SEQ ID NO: 9 - protein, 27 aa, Conus purpurascens
  PEPTIDE (1)..(27): Xaa is Hyp
CKIXNQKCFQHLDDCCSRKCNRFNKCV

SEQ ID NO: 10 - protein, 27 aa, Conus purpurascens
  PEPTIDE (1)..(27): Xaa is Hyp
CRIXNQACFQHLDDCCSRKCNRFNKCV

SEQ ID NO: 11 - protein, 27 aa, Conus purpurascens
  PEPTIDE (1)..(27): Xaa is Hyp
CRIXNQKCMQHLDDCCSRKCNRFNKCV

SEQ ID NO: 12 - protein, 27 aa, Conus purpurascens
  PEPTIDE (1)..(27): Xaa is Hyp
CRIXNQKCYQHLDDCCSRKCNRFNKCV

SEQ ID NO: 13 - protein, 27 aa, Conus purpurascens
  PEPTIDE (1)..(27): Xaa is Hyp
CQIXNQKCFQHLDDCCSRKCNRFNKCV

SEQ ID NO: 14 - protein, 27 aa, Conus purpurascens
  PEPTIDE (1)..(27): Xaa is Hyp
CRIXNQKCFQALDDCCSRKCNRFNKCV

SEQ ID NO: 15 - protein, 27 aa, Conus purpurascens
  PEPTIDE (1)..(27): Xaa is Hyp
CRIXNQKCFQHLDACCSRKCNRFNKCV

SEQ ID NO: 16 - protein, 27 aa, Conus purpurascens
  PEPTIDE (1)..(27): Xaa is Hyp
CRIXNAKCFQHLDDCCSRKCNRFNKCV

SEQ ID NO: 17 - protein, 27 aa, Conus purpurascens
  PEPTIDE (1)..(27): Xaa is Hyp
CRIXNQKCFQHLDDCCSRKCARFNKCV

SEQ ID NO: 18 - protein, 27 aa, Conus purpurascens
  PEPTIDE (1)..(27): Xaa is Hyp
CRIXNQKCFQHLDDCCARKCNRFNKCV

SEQ ID NO: 19 - protein, 27 aa, Conus purpurascens
  PEPTIDE (1)..(27): Xaa is Hyp
CRIXNQKCFQHLDDCCSRKCNRFAKCV

SEQ ID NO: 20 - protein, 27 aa, Conus purpurascens
  PEPTIDE (1)..(27): Xaa is Hyp
CRIXNQKCFQHADDCCSRKCNRFNKCV

SEQ ID NO: 21 - protein, 27 aa, Conus purpurascens
  PEPTIDE (1)..(27): Xaa is Hyp
CRIXNQKCFQHLADCCSRKCNRFNKCV

SEQ ID NO: 22 - protein, 27 aa, Conus purpurascens
  PEPTIDE (1)..(27): Xaa is Hyp
CRIXNQKCFAHLDDCCSRKCNRFNKCV

SEQ ID NO: 23 - protein, 27 aa, Conus purpurascens
  PEPTIDE (1)..(27): Xaa is Hyp
CRIXNQKCFQHLDDCCSRKCNRFNKCA

SEQ ID NO: 24 - protein, 27 aa, Conus purpurascens
CRIANQKCFQHLDDCCSRKCNRFNKCV

SEQ ID NO: 25 - protein, 27 aa, Conus purpurascens
  PEPTIDE (1)..(27): Xaa is Hyp
CRIXAQKCFQHLDDCCSRKCNRFNKCV

SEQ ID NO: 26 - protein, 27 aa, Artificial
  generic conopeptide sequence
XCXXXGXXCXXXQXDCCNXTCTXSXCX

SEQ ID NO: 27 - protein, 27 aa, Artificial
  generic conopeptide sequence
XCXTXGXXCXXXQXDCCGXACIITICX

SEQ ID NO: 28 - protein, 27 aa, Artificial
  generic conopeptide sequence
XCXXTGXXCXXXQXDCCGXACIITICX

(19) United States
(12) Patent Application Publication
(10) Pub. No.: US 2003/0224343 A1
(43) Pub. Date: Dec. 4, 2003

(54) Kappa-PVIIA-related conotoxins as organ protectants

(75) Inventors: Karen Pemberton-Goodman, Guilford, CT (US); Robert M. Jones, San Diego, CA (US); Davis L. Temple JR., Wallingford, CT (US); J. Michael McIntosh, Salt Lake City, UT (US); Baldomero M. Olivera, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignees: University of Utah Research Foundation, Salt Lake City, UT; Cognetix, Inc., Salt Lake City, UT

(21) Appl. No.: 10/352,254

(22) Filed: Jan. 28, 2003

Related U.S. Application Data
(60) Provisional application No. 60/352,219, filed on Jan. 29, 2002.

(51) Int. Cl. A01N 1/02; A61K 38/16

(52) U.S. Cl. 435/1.1; 514/2

(57) ABSTRACT

The invention relates to κ-PVIIA-related conotoxins and their use as organ protecting agents, i.e., organ protectants. These conotoxins can be used for arresting, protecting or preserving an organ, such as a circulatory organ, a respiratory organ, a urinary organ, a digestive organ, a reproductive organ, an endocrine organ or a neurological organ. These conotoxins can also be used for arresting, protecting or preserving somatic cells.

### CROSS-REFERENCE TO RELATED APPLICATION

The present application is related to and claims priority under 35 USC §119(e) to U.S. provisional patent application Serial No. 60/352,219 filed on Jan. 29, 2002, incorporated herein by reference.

This invention was made in part with Government support under SBIR Phase I Grant No. R43 HL65793-01 awarded by the National Institutes of Health, Bethesda, Md. The United States Government has certain rights in the invention.

### BACKGROUND OF THE INVENTION

The invention relates to κ-PVIIA-related conotoxins and pharmaceutically acceptable salts thereof and their use as organ protecting agents, i.e., organ protectants. These conotoxins can be used for arresting, protecting or preserving an organ, such as a circulatory organ, a respiratory organ, a urinary organ, a digestive organ, a reproductive organ, an endocrine organ or a neurological organ. These conotoxins can also be used for arresting, protecting or preserving somatic cells.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

κ-PVIIA, a 27 amino acid peptide that was originally purified from the venom of the purple cone snail Conus purpurascens (Terlau et al., 1996; U.S. Pat. No. 5,672,682), has been previously identified as a potent antagonist of the Shaker H4 potassium channel (IC₅₀˜60 nM). In the same study, no detectable activity on the voltage-gated potassium channels Kv1.1 or Kv1.4 (Terlau et al., 1996) was noted. Chimeras constructed from the Shaker and the Kv1.1 K⁺ channels have identified the putative pore-forming region between the fifth and sixth transmembrane region as the site of the toxin sensitivity (Shon et al., 1998). It appears that κ-PVIIA interacts with the external tetraethyl-ammonium binding site on the Shaker channel. Although both κ-PVIIA and charybdotoxin inhibit the Shaker channel, they must interact differently. The F425G Shaker mutation increases charybdotoxin affinity by three orders of magnitude but abolishes κ-PVIIA sensitivity (Shon et al., 1998). κ-PVIIA appears to block the ion pore with a 1:1 stoichiometry, and its binding to open or closed channels is very different (Terlau et al., 1999). Chronically applied to whole oocytes or outside-out patches, κ-PVIIA inhibition appears as a voltage-dependent relaxation in response to the depolarizing pulse used to activate the channels (Garcia et al., 1999).

Potassium channels are vital in controlling the resting membrane potential in excitable cells and can be broadly subdivided into three classes, voltage-gated K⁺ channels, Ca²⁺ activated K⁺ channels and ATP-sensitive K⁺ channels (K_{ATP} channels). ATP-sensitive potassium channels were originally described in cardiac tissue (Noma, 1983). In subsequent years they have also been identified in pancreatic cells, skeletal, vascular and neuronal tissue. This group of K⁺ channels is modulated by intracellular ATP levels and as such, couples cellular metabolism to electrical activity. Enhanced levels of ATP result in closure of the K_{ATP} channels. The K_{ATP} channel is thought to be an octomeric complex comprised of two different subunits in a 1:1 stoichiometry; a weakly inward rectifying K⁺ channel Kir6.x (6.1 or 6.2), which is thought to form the channel pore, and a sulphonylurea (SUR) subunit. So far, three variants of the SUR have been identified: SUR1, SUR2A and SUR2B. While the Kir6.2 subunit is common to K_{ATP} channels in cardiac, pancreatic and neuronal tissue (Kir6.1 is preferentially expressed in vascular smooth muscle tissue), the SUR is differentially expressed. Kir6.2/SUR1 reconstitute the neuronal/pancreatic beta-cell K_{ATP} channel, whereas Kir6.2/SUR2A are proposed to reconstitute the cardiac K_{ATP} channels.

Potassium channels comprise a large and diverse group of proteins that, through maintenance of the cellular membrane potential, are fundamental in normal biological function. The potential therapeutic applications for compounds that open K⁺ channels are far-reaching and include treatments of a wide range of disease and injury states, including cerebral and cardiac ischemia and asthma. Recently, considerable interest has focused around the ability of K⁺ channel openers to produce relaxation of airway smooth muscle, and as such, these compounds may offer a novel approach to the treatment of bronchial asthma (Lin et al., 1998; Muller-Schweinitzer and Fozard, 1997; Morley, 1994; Barnes, 1992). Furthermore, the cardioprotective effects of K⁺ channel openers are now well established in experimental animal models of cardiac ischemia (Grover, 1996; Jung et al., 1998; Kouchi et al., 1998). Less is known about the ability of these compounds to limit neuronal damage caused from cerebral ischemia. Most progress in the treatment of cerebral ischemia has focused around the development of compounds to reduce the influx of sodium and calcium ions. K⁺ channel openers, which restore the resting membrane potential, could also be employed to reduce acute damage associated with an ischemic episode in neuronal tissue (Reshef et al., 1998; Wind et al., 1997), as well as reducing glutamate-induced excitotoxicity (Lauritzen et al., 1997). However, clinical use of K_{ATP} openers has been somewhat limited due to their cardiovascular side effects (i.e., drop in blood pressure).

Thus, it is desired to develop new agents for opening ATP-sensitive potassium channels which can be used as organ protecting agents.

### SUMMARY OF THE INVENTION

The invention relates to κ-PVIIA-related conotoxins and pharmaceutically acceptable salts thereof and their use as organ protecting agents, i.e., organ protectants. These conotoxins can be used for arresting, protecting or preserving an organ, such as a circulatory organ, a respiratory organ, a urinary organ, a digestive organ, a reproductive organ, an endocrine organ or a neurological organ. These conotoxins can also be used for arresting, protecting or preserving somatic cells.

In accordance with the present invention, κ-PVIIA-related conotoxins refer to the conotoxins κ-PVIIA, E6.2, P6.1, P6.3, congeners thereof, analogs thereof or derivatives thereof. These peptides have been found to have organ protecting activity.

In one embodiment, the present invention provides a method for arresting, preserving or protecting an organ by administering a therapeutically effective amount of a κ-PVIIA-related conotoxin or pharmaceutically acceptable salt thereof. As used herein, the term “arresting” shall mean the act of stopping as in the act of stopping the pathological process resulting from myocardial ischemia. The term “preserving” shall mean the act of keeping alive or keeping safe from harm or injury The term “protecting” shall mean the act of affording defense against a deleterious influence such as the pathological process resulting from myocardial ischemia.

In a second embodiment, the present provides a method for arresting, preserving or protecting an organ by administering a therapeutically effective amount of a κ-PVIIA-related conotoxin or pharmaceutically acceptable salt thereof in combination with an adenosine receptor agonist (A1, A2a or A3).

In a third embodiment, the present provides a method for arresting, preserving or protecting an organ by administering a therapeutically effective amount of a κ-PVIIA-related conotoxin or pharmaceutically acceptable salt thereof in combination with an adenosine receptor agonist and a local anesthetic.

In a fourth embodiment, the present provides a method for arresting, preserving or protecting an organ by administering a therapeutically effective amount of a κ-PVIIA-related conotoxin or pharmaceutically acceptable salt thereof in combination with a potassium channel opener or agonist and optionally an atrioventricular (AV) blocker.

In a fifth embodiment, a hemostatic agent is also administered to an individual receiving any of the above treatments. Such a hemostatic agent may be a “clot buster” agent, a thrombolytic agent, an anti-coagulant agent or an anti-platelet aggregation agent.

In accordance with the present invention, suitable organs which can be protected include a circulatory organ, a respiratory organ, a urinary organ, a digestive organ, a reproductive organ, an endocrine organ or a neurological organ. Somatic cells can also be protected by the present method. Unless dictated otherwise by the context of its usage, the term “protect” is intended to include “arrest” and “preserve” as used herein.

In a particularly preferred embodiment, the organ is the heart. The method can be used to arrest, protect or preserve the heart during open heart surgery, angioplasty, valve surgery, transplantation or cardiovascular disease so as to reduce heart damage before, during or following cardiovascular intervention or to protect from damage those portions of the heart that have been starved of normal flow of blood, nutrients or oxygen, such as in reperfusion injury.

### BRIEF DESCRIPTION OF THE FIGURES

FIG. 1 shows fluorimetry measurements of intracellular K⁺ (determined with PBFI dye) following exposure to increasing concentrations of κ-PVIIA in primary cultures of ventricular myocytes. The data shown is from one trial and is represented as mean change in fluorescence ±S.E.M. (*p<0.05, unpaired t-test).

FIGS. 2A-2B show fluorimetry measurements of membrane potential (determined with Di-8-ANEPPs dye) following exposure to increasing concentrations of κ-PVIIA in primary cultures of ventricular myocytes (FIG. 2A) or cortex (FIG. 2B). Cells were loaded into 96 well plates at least six days before the experiment. Results are expressed as Mean±SEM and represent average data from between two and five individual trials.

FIGS. 3A-3B are bar graphs showing the inhibition of the κ-PVIIA (100 nM) response with 10 nM Glibenclamide (Glib) in primary cultures of myocytes (FIG. 3A) or with 50 uM Tolbutamide (Tolb) in primary cultures of cortex (FIG. 3B). Data represents mean±S.E.M.

FIGS. 4A-4C are whole cell recordings showing currents elicited by κ-PVIIA in (FIG. 4A) cortical cells and (FIG. 4B) myocytes. FIG. 4C shows I-V relationship of κ-PVIIA-induced current from a cardiac myocyte.

FIG. 5 is a bar graph showing the protective effect of 10 nM κ-PVIIA against hypoxia induced depolarization. Bars represent Mean±S.E.M.

FIG. 6 shows the effect of increasing concentrations of κ-PVIIA on glutamate-induced (100 uM) excitotoxicity measured six hours following glutamate washout (three to six trials).

FIG. 7 shows the infarct size as a % of the risk region (ischemic zone) as plotted for the six groups studied. Open symbols indicate individual experiments and solid symbols indicated group means. Triangles indicate animals receiving drug 5 min prior to reperfusion and indicate animals receiving drug 10 min after reperfusion.

FIG. 8 is a plot of infarct size vs. risk zone size. Solid circles indicate the plot for the untreated controls and the line is the regression for that group. Protected groups all lie below the line as indicated by the open symbols.

FIG. 9 is a graph showing κ-PVIIA (CGX-1051) induced reduction in infarct size expressed as a percentage of the area at risk in the canine AMI model. Data represents mean+/−SEM from 6 dogs per dose. CON-Control, OCC(30′)—30 min after occlusion, DRUG-Immediately following drug administration, REP1, REP2, REP3-1, 2 and 3 hours following reperfusion.

FIGS. 10A and 10B are graphs showing the lack of effect of any of the examined doses of κ-PVIIA (CGX-1051) on blood pressure (FIG. 10A) and heart rate (FIG. 10B).

FIG. 11 is a graph showing reduction in incidence of ventricular fibrillation following administration of κ-PVIIA (CGX-1051).

### DETAILED DESCRIPTION OF THE PREFERRED EMBODIMENT

The invention relates to κ-PVIIA-related conotoxins and pharmaceutically acceptable salts thereof and their use as organ protecting agents, i.e., organ protectants. These conotoxins can be used for arresting, protecting or preserving an organ, such as a circulatory organ, a respiratory organ, a urinary organ, a digestive organ, a reproductive organ, an endocrine organ or a neurological organ. These conotoxins can also be used for arresting, protecting or preserving somatic cells.

For purposes of the present invention, κ-PVIIA refers to a peptide having the following general formula:

Cys-Xaa₁-Ile-Xaa₂-Asn-Gln-Xaa₃-Cys-Xaa₄-Gln-Xaa₅-Leu-Asp-Asp-Cys-Cys-Ser-Xaa₁-Xaa₃-Cys-Asn-Xaa₁-Xaa₄-Asn-Xaa₃-Cys-Val (SEQ ID NO:1), wherein Xaa₁ and Xaa₃ are independently Arg, homoarginine, ornithine, Lys, N-methyl-Lys, N,N-dimethyl-Lys, N,N,N-trimethyl-Lys, any synthetic basic amino acid, His or halo-His; Xaa₂ is Pro or hydroxy-Pro (Hyp); Xaa₄ is Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Tip, halo-Trp (D or L) or any synthetic aromatic amino acid; and Xaa₅ is His or halo-His. The C-terminus may contain a free carboxyl group or an amide group. The halo is preferably bromine, chlorine or iodine. It is preferred that Xaa₁ is Arg and Xaa₅ is His. It is more preferred that Xaa₁ is Arg, Xaa₃ is Lys, Xaa₄ is Phe and Xaa₅ is His. It is further preferred that the C-terminus contains a free carboxyl group.

For purposes of the present invention, E6.2 refers to a peptide having the following general formula:

Xaa₂-Cys-Xaa₃-Xaa₂-Xaa₃-Gly-Xaa₁-Xaa₃-Cys-Xaa₄-Xaa₂-Xaa₅-Gln-Xaa₃-Asp-Cys-Cys-Asn-Xaa₃-Thr-Cys-Thr-Xaa₁-Ser-Xaa₃-Cys-Xaa₂ (SEQ ID NO:26), wherein Xaa₁, Xaa₂, Xaa₃, Xaa₄ and Xaa₅ is as defined above. The C-terminus may contain a free carboxyl group or an amide group, preferably a free carboxyl. It is preferred that Xaa₁ is Arg, Xaa₃ is Lys, Xaa₄ is Phe and Xaa₅ is His. It is more preferred that Xaa₁ is Arg, Xaa₂ is Pro, Xaa₃ is Lys, Xaa₄ is Phe and Xaa₅ is His.

For purposes of the present invention, P6.1 refers to a peptide having the following general formula:

Xaa₂-Cys-Xaa₃-Thr-Xaa₂-Gly-Xaa₁-Xaa₃-Cys-Xaa₄-Xaa₂-Xaa₅-Gln-Xaa₃-Asp-Cys-Cys-Gly-Xaa₁-Ala-Cys-Ile-Ile-Thr-Ile-Cys-Xaa₂ (SEQ ID NO:27), wherein Xaa₁, Xaa₂, Xaa₃, Xaa₄ and Xaa₅ is as defined above. The C-terminus may contain a free carboxyl group or an amide group, preferably a free carboxyl. It is preferred that Xaa₁ is Arg, Xaa₃ is Lys, Xaa₄ is Phe and Xaa₅ is His. It is more preferred that Xaa₁ is Arg, Xaa₂ is Hyp except at the C-terminus which is Pro, Xaa₃ is Lys, Xaa₄ is Phe and Xaa₅ is His.

For purposes of the present invention, P6.3 refers to a peptide having the following general formula:

Xaa₂-Cys-Xaa₃-Xaa₃-Thr-Gly-Xaa₁-Xaa₃-Cys-Xaa₄-Xaa₂-Xaa₅-Gln-Xaa₃-Asp-Cys-Cys-Gly-Xaa₁-Ala-Cys-Ile-Ile-Thr-Ile-Cys-Xaa₂ (SEQ ID NO:28), wherein Xaa₁, Xaa₂, Xaa₃, Xaa₄ and Xaa₅ is as defined above. The C-terminus may contain a free carboxyl group or an amide group, preferably a free carboxyl. It is preferred that Xaa₁ is Arg, Xaa₃ is Lys, Xaa₄ is Phe and Xaa₅ is His. It is more preferred that Xaa₁ is Arg, Xaa₂ is Pro, Xaa₃ is Lys, Xaa₄ is Phe and Xaa₅ is His.

The κ-PVIIA analogs refer to peptides having the following formulas:

| | | |
|---|---|---|
| κ-PVIIA[R18A]: | Cys-Arg-Ile-Hyp-Asn-Gln-Lys-Cys-Phe-Gln-His-Leu-Asp-Asp-Cys- | (SEQ ID NO:2) |
| | Cys-Ser-Ala-Lys-Cys-Asn-Arg-Phe-Asn-Lys-Cys-Val; | |
| κ-PVIIA[R22A]: | Cys-Arg-Ile-Hyp-Asn-Gln-Lys-Cys-Phe-Gln-His-Leu-Asp-Asp-Cys- | (SEQ ID NO:3) |
| | Cys-Ser-Arg-Lys-Cys-Asn-Ala-Phe-Asn-Lys-Cys-Val; | |
| κ-PVIIA[13A]: | Cys-Arg-Ala-Hyp-Asn-Gln-Lys-Cys-Phe-Gln-His-Leu-Asp-Asp-Cys- | (SEQ ID NO:4) |
| | Cys-Ser-Arg-Lys-Cys-Asn-Arg-Phe-Asn-Lys-Cys-Val; | |
| κ-PVIIA[K19A]: | Cys-Arg-Ile-Hyp-Asn-Gln-Lys-Cys-Phe-Gln-His-Leu-Asp-Asp-Cys- | (SEQ ID NO:5) |
| | Cys-Ser-Arg-Ala-Cys-Asn-Arg-Phe-Asn-Lys-Cys-Val; | |
| κ-PVIIA[R2A]: | Cys-Ala-Ile-Hyp-Asn-Gln-Lys-Cys-Phe-Gln-His-Leu-Asp-Asp-Cys- | (SEQ ID NO:6) |
| | Cys-Ser-Arg-Lys-Cys-Asn-Arg-Phe-Asn-Lys-Cys-Val; | |
| κ-PVIIA[F9A]: | Cys-Arg-Ile-Hyp-Asn-Gln-Lys-Cys-Ala-Gln-His-Leu-Asp-Asp-Cys- | (SEQ ID NO:7) |
| | Cys-Ser-Arg-Lys-Cys-Asn-Arg-Phe-Asn-Lys-Cys-Val; | |
| κ-PVIIA[K25A]: | Cys-Arg-Ile-Hyp-Asn-Gln-Lys-Cys-Phe-Gln-His-Leu-Asp-As-Cys- | (SEQ ID NO:8) |
| | Cys-Ser-Arg-Lys-Cys-Asn-Arg-Phe-Asn-Ala-Cys-Val; | |
| κ-PVIIA[R2K]: | Cys-Lys-Ile-Hyp-Asn-Gln-Lys-Cys-Phe-Gln-His-Leu-Asp-Asp-Cys- | (SEQ ID NO:9) |
| | Cys-Ser-Arg-Lys-Cys-Asn-Arg-Phe-Asn-Lys-Cys-Val; | |
| κ-PVIIA[K7A]: | Cys-Arg-Ile-Hyp-Asn-Gln-Ala-Cys-Phe-Gln-His-Leu-Asp-Asp-Cys- | (SEQ ID NO:10) |
| | Cys-Ser-Arg-Lys-Cys-Asn-Arg-Phe-Asn-Lys-Cys-Val; | |
| κ-PVIIA[F9M]: | Cys-Arg-Ile-Hyp-Asn-Gln-Lys-Cys-Met-Gln-His-Leu-Asp-Asp-Cys- | (SEQ ID NO:11) |
| | Cys-Ser-Arg-Lys-Cys-Asn-Arg-Phe-Asn-Lys-Cys-Val; | |
| κ-PVIIA[F9Y]: | Cys-Arg-Ile-Hyp-Asn-Gln-Lys-Cys-Tyr-Gln-His-Leu-Asp-Asp-Cys- | (SEQ ID NO:12) |
| | Cys-Ser-Arg-Lys-Cys-Asn-Arg-Phe-Asn-Lys-Cys-Val; | |
| κ-PVIIA[R2Q]: | Cys-Gln-Ile-Hyp-Asn-Gln-Lys-Cys-Phe-Gln-His-Leu-Asp-Asp-Cys- | (SEQ ID NO:13) |
| | Cys-Ser-Arg-Lys-Cys-Asn-Arg-Phe-Asn-Lys-Cys-Val; | |
| κ-PVIIA[H11A]: | Cys-Arg-Ile-Hyp-Asn-Gln-Lys-Cys-Phe-Gln-Ala-Leu-Asp-Asp-Cys- | (SEQ ID NO:14) |
| | Cys-Ser-Arg-Lys-Cys-Asn-Arg-Phe-Asn-Lys-Cys-Val; | |
| κ-PVIIA[D14A]: | Cys-Arg-Ile-Hyp-Asn-Gln-Lys-Cys-Phe-Gln-His-Leu-Asp-Ala-Cys- | (SEQ ID NO:15) |
| | Cys-Ser-Arg-Lys-Cys-Asn-Arg-Phe-Asn-Lys-Cys-Val; | |
| κ-PVIIA[Q6A]: | Cys-Arg-Ile-Hyp-Asn-Ala-Lys-Cys-Phe-Gln-His-Leu-Asp-Asp-Cys- | (SEQ ID NO:16) |
| | Cys-Ser-Arg-Lys-Cys-Asn-Arg-Phe-Asn-Lys-Cys-Val; | |
| κ-PVIIA[N21A]: | Cys-Arg-Ile-Hyp-Asn-Gln-Lys-Cys-Phe-Gln-His-Leu-Asp-Asp-Cys- | (SEQ ID NO:17) |
| | Cys-Ser-Arg-Lys-Cys-Ala-Arg-Phe-Asn-Lys-Cys-Val; | |
| κ-PVIIA[S17A]: | Cys-Arg-Ile-Hyp-Asn-Gln-Lys-Cys-Phe-Gln-His-Leu-Asp-Asp-Cys- | (SEQ ID NO:18) |
| | Cys-Ala-Arg-Lys-Cys-Asn-Arg-Phe-Asn-Lys-Cys-Val; | |
| κ-PVIIA[N42A]: | Cys-Arg-Ile-Hyp-Asn-Gln-Lys-Cys-Phe-Gln-His-Leu-Asp-Asp-Cys- | (SEQ ID NO:19) |
| | Cys-Ser-Arg-Lys-Cys-Asn-Arg-Phe-Ala-Lys-Cys-Val; | |
| κ-PVIIA[L12A]: | Cys-Arg-Ile-Hyp-Asn-Gln-Lys-Cys-Phe-Gln-His-Ala-Asp-Asp-Cys- | (SEQ ID NO:20) |
| | Cys-Ser-Arg-Lys-Cys-Asn-Arg-Phe-Asn-Lys-Cys-Val; | |
| κ-PVIIA[D13A]: | Cys-Arg-Ile-Hyp-Asn-Gln-Lys-Cys-Phe-Gln-His-Leu-Ala-Asp-Cys- | (SEQ ID NO:21) |
| | Cys-Ser-Arg-Lys-Cys-Asn-Arg-Phe-Asn-Lys-Cys-Val; | |
| κ-PVIIA[Q10A]: | Cys-Arg-Ile-Hyp-Asn-Gln-Lys-Cys-Phe-Ala-His-Leu-Asp-Asp-Cys- | (SEQ ID NO:22) |
| | Cys-Ser-Arg-Lys-Cys-Asn-Arg-Phe-Asn-Lys-Cys-Val; | |
| κ-PVIIA[V27A]: | Cys-Arg-Ile-Hyp-Asn-Gln-Lys-Cys-Phe-Gln-His-Leu-Asp-Asp-Cys- | (SEQ ID NO:23) |
| | Cys-Ser-Arg-Lys-Cys-Asn-Arg-Phe-Asn-Lys-Cys-Ala | |
| κ-PVIIA[Q4A]: | Cys-Arg-Ile-Ala-Asn-Gln-Lys-Cys-Phe-Gln-His-Leu-Asp-Asp-Cys- | (SEQ ID NO:24) |
| | Cys-Ser-Arg-Lys-Cys-Asn-Arg-Phe-Asn-Lys-Cys-Val and | |
| κ-PVIIA[N5A]: | Cys-Arg-Ile-Hyp-Ala-Gln-Lys-Cys-Phe-Gln-His-Leu-Asp-Asp-Cys- | (SEQ ID NO:25) |
| | Cys-Ser-Arg-Lys-Cys-Asn-Arg-Phe-Asn-Lys-Cys-Val. | |

It is preferred that the C-terminus contains a free carboxyl group.

The present invention further relates to derivatives of the above peptides or analogs. In accordance with the present invention, derivatives include peptides or analogs in which the Arg residue may be substituted by Lys, ornithine, homoarginine, nor-Lys, N-methyl-Lys, N,N-dimethyl-Lys, N,N,N-trimethyl-Lys or any synthetic basic amino acid; the Xaa₁ residues may be substituted by Arg, ornithine, homoarginine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated. The Cys residues may be in D or L configuration and may optionally be substituted with homocysteine (D or L). The Tyr residues may also be substituted with ¹²⁵I-Tyr or with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂ up to and including n=8. The Leu residues may be substituted with Leu (D). The Gla residues may be substituted with Glu.

The present invention is further directed to derivatives of the above peptides and peptide derivatives which are cyclic permutations in which the cyclic permutants retain the native bridging pattern of native toxin. See Craik et al. (2001).

Examples of synthetic aromatic amino acid include, but are not limited to, nitro-Phe, 4-substituted-Phe wherein the substituent is C₁-C₃ alkyl, carboxyl, hydroxymethyl, sulphomethyl, halo, phenyl, —CHO, —CN, —SO₃H and —NHAc. Examples of synthetic hydroxy containing amino acid, include, but are not limited to, 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference, and such as shown in the following schemes 1-3.

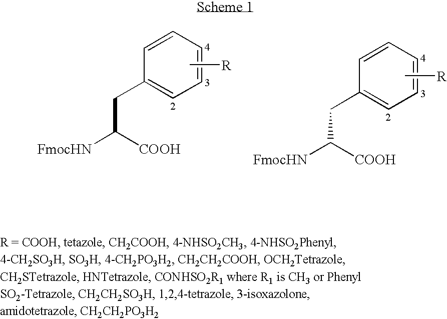

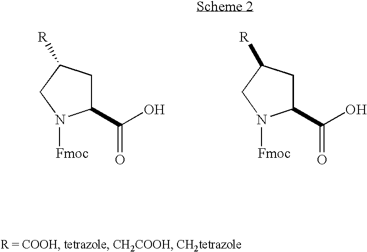

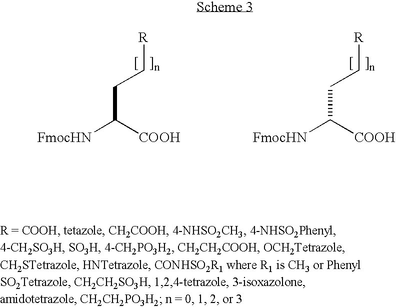

Optionally, in the peptides and analogs described above, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N—, S— or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include, but are not limited to, D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The glycan may also include similar polyhydroxy groups, such as D-penicillamine 2,5 and halogenated derivatives thereof or polypropylene glycol derivatives. The glycosidic linkage is beta and 1→4 or 1→3, preferably 1→3. The linkage between the glycan and the amino acid may be alpha or beta, preferably alpha and is 1→.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the core glycans, of which eight have been identified. The type of glycosidic linkage (orientation and connectivities) are defined for each core glycan. Suitable glycans and glycan analogs are described further in U.S. Ser. No. 09/420,797, filed Oct. 19, 1999 and in PCT Application No. PCT/US99/24380, filed Oct. 19, 1999 (PCT Published Application No. WO 00/23092), each incorporated herein by reference. A preferred glycan is Gal(β1→3)GalNAc(α1→).

Optionally, in the above peptides, pairs of Cys residues may be replaced pairwise with isosteric lactam or ester-thioether replacements, such as Ser/(Glu or Asp), Lys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues. In addition, individual Cys residues may be replaced with homoCys, seleno-Cys or penicillamine, so that disulfide bridges may be formed between Cys-homoCys or Cys-penicillamine, or homoCys-penicillamine and the like.

The present invention, in another aspect, relates to a pharmaceutical composition comprising an effective amount of κ-PVIIA-related conotoxins. Such a pharmaceutical composition has the capability of acting as organ protecting agents, i.e., organ protectants. These conotoxins can be used for arresting, protecting or preserving an organ, such as a circulatory organ, a respiratory organ, a urinary organ, a digestive organ, a reproductive organ, an endocrine organ or a neurological organ.

The κ-PVIIA-related conotoxins can be isolated from Conus such as described in U.S. Pat. No. 5,672,682 for κ-PVIIA from Conus purpurascens, or it can be chemically synthesized by general synthetic methods such as described in U.S. Pat. No. 5,672,682. Alternatively, the native peptide can be synthesized by conventional recombinant DNA techniques (Sambrook et al., 1989) using the DNA encoding the conotoxin, such as DNA encoding κ-PVIIA (Shon et al., 1998) or DNA encoding E6.2, P6.1 or P6.3 as described in U.S. patent application Ser. No. 09/910,082 and international patent application No. PCT/US01/23041, each incorporated herein by reference. The peptides are also synthesized using an automated synthesizer. Amino acids are sequentially coupled to an MBHA Rink resin (typically 100 mg of resin) beginning at the C-terminus using an Advanced ChemTech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopropylethylamine (DIEA). The FMOC protecting group is removed by treatment with a 20% solution of piperidine in dimethylformamide(DMF). Resins are subsequently washed with DMF (twice), followed by methanol and NMP.

Muteins, analogs or active fragments, of the foregoing κ-PVIIA-related conotoxin peptides are also contemplated here. See, e.g., Hammerland et al (1992). Derivative muteins, analogs or active fragments of the conotoxin peptides may be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 22, line 33); and U.S. Pat. No. 5,364,769 (see particularly col. 4, line 55 to col. 7, line 26), each incorporated herein by reference.

In accordance with the present invention, κ-PVIIA-related conotoxins and pharmaceutically acceptable salts thereof are used for arresting, protecting or preserving an organ. The organ may be intact in the subject or may have been isolated (such as for transplantation). The organ may be a circulatory organ, a respiratory organ, a urinary organ, a digestive organ, a reproductive organ, an endocrine organ or a neurological organ. The present invention is particularly useful for arresting, protecting or preserving the heart during open heart surgery, angioplasty, valve surgery, bypass surgery, transplantation, or cardiovascular disease so as to reduce heart damage before, during or following cardiovascular intervention or to protect those portions of the heart that have been starved of normal flow of blood, nutrients and/or oxygen (reperfusion injury). The present invention is also particularly useful for cardioplegia, which is a technique of myocardial preservation during cardiac surgery, usually employing infusion of a cold, potassium laced solution, sometimes fixed with blood, to achieve arrest of the myocardial fibers and to reduce their oxygen consumption to nearly nothing. Techniques using warm (body temperature) blood can also be used with the present κ-PVIIA-related conotoxins and pharmaceutically acceptable salts thereof.

The κ-PVIIA-related conotoxins and pharmaceutically acceptable salts thereof can be used in conjunction with other agents for arresting, protecting or preserving organs in accordance with the present invention. Thus, κ-PVIIA-related conotoxins and pharmaceutically acceptable salts thereof can be coadministered with an adenosine receptor agonist, a local anesthetic, a potassium channel opener or agonist, an AV blocker, and/or a hemostatic agent. Examples of adenosine receptor agonists include, but are not limited to, A1, A2a and A3 agents. A1 agents include, but are not limited to, CPA, NECA, CGS-21680, AB-MECA, AMP579, 9APNEA, CHA, ENBA. A2a agents include, but are not limited to, R-PIA, DPMA, CGS-21680, ATL146e. A3 agents include, but are not limited to, CCPA, CI-IB-MECA, IB-MECA. Suitable local anesthetics include, but are not limited to, mexilitine, diphenylhydantoin, prilocaine, procaine, mipivicaine, bupivicaine, lidocaine and class 1B anti-arrhythmic agents, i.e. lignocaine. Suitable potassium channel openers or agonists include, but are not limited to, cromakalin, pinacidil, nicorandil, NS-1619, diazoxide and minoxidil. Suitable AV blockers include, but are not limited to, verapamil. Hemostatic agents may be a “clot buster” agent, a thrombolytic agent, an anti-coagulant agent or an anti-platelet aggregation agent. Suitable “clot buster” agents include, but are not limited to, streptokinase, urokinase and ACTIVASE. Suitable thrombolytic agents include, but are not limited to, streptokinase, urokinase, alteplase, reteplase and tenecteplase. Suitable anti-coagulant agents include, but are not limited to, heparin, enoxaparin and dalteparin. Suitable anti-platelet aggregation agents include, but are not limited to, aspirin, clopidogrel, abciximab, eptifibatide and tirofiban.

The κ-PVIIA-related conotoxins and pharmaceutically acceptable salts thereof disclosed herein can also be used for the treatment of arrhythmia, urinary incontinence, angina, reperfusion injury, diabetes, retinopathy, neuropathy, nephropathy, peripheral circulation disturbances, acute heart failure, hypertension, cerebral vasospasm accompanying subarachnoid hemorrhage, anxiety disorder, cerebral ischemia, coronary artery bypass graft (CABG) surgery, ischemic heart disease and congestive heart failure. The κ-PVIIA-related conotoxins and pharmaceutically acceptable salts thereof disclosed herein can also be used for open heart surgery, bypass surgery, heart transplant surgery and cardioplegia. Cardioplegia is a technique of myocardial preservation during cardiac surgery usually employing infusion of a cold, potassium laced solution, sometimes fixed with blood, to achieve arrest of the myocardial fibers and reduce their oxygen consumption to nearly nothing. Techniques using warm (body temperature) blood are also used.

Pharmaceutical compositions containing a compound of the present invention or its pharmaceutically acceptable salts as the active ingredient can be prepared according to conventional pharmaceutical compounding techniques. See, for example, Remington's Pharmaceutical Sciences, 18th Ed. (1990, Mack Publishing Co., Easton, Pa.). Typically, an ATP-sensitive potassium channel opening amount of the active ingredient will be admixed with a pharmaceutically acceptable carrier. The carrier may take a wide variety of forms depending on the form of preparation desired for administration, e.g., intravenous, oral or parenteral. The compositions may further contain antioxidizing agents, stabilizing agents, preservatives and the like. For examples of delivery methods, see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable for passage through the gastrointestinal tract, while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, stabilizing agents, buffers and the like. One particularly suitable stabilizing agent for the conotoxin peptides contemplated here is carboxymethyl cellulose. This agent may be particularly effective due to the excess positive charge of the contemplated conotoxin peptides. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:

(a) pump (see, e.g., Lauer & Hatton (1993), Zimm et al. (1984), Ettinger et al. (1978) and cardioplegia system of Medtronic, Inc.);

(b), microencapsulation (see, e.g., U.S. Pat. Nos. 4,352,883; 4,353,888; and 5,084,350);

(c) continuous release polymer implants (see, e.g., U.S. Pat. No. 4,883,666);

(d) macroencapsulation (see, e.g., U.S. Pat. Nos. 5,284,761, 5,158,881, 4,976,859 and 4,968,733 and published PCT patent applications WO 92/19195, WO 95/05452);

(e) naked or unencapsulated cell grafts to the CNS (see, e.g., U.S. Pat. Nos. 5,082,670 and 5,618,531);

(f) injection, either subcutaneously, intravenously, intra-arterially, intramuscularly, or to other suitable site; or

(g) oral administration, in capsule, liquid, tablet, pill, or prolonged release formulation.

In one embodiment of this invention, an active agent is delivered directly into the CNS, preferably to the brain ventricles (e.g. i.c.v.), brain parenchyma, the intrathecal space or other suitable CNS location, most preferably intrathecally.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in a therapeutically effective amount. By a “therapeutically effective amount” or simply “effective amount” of an active compound is meant a sufficient amount of the compound to arrest, preserve or protect an organ at a reasonable benefit/risk ratio applicable to any medical treatment. The actual amount administered, and the rate and time-course of administration, will depend on the nature and severity of the condition being treated. The administration may be continuous or be intermittent. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Dosage may be adjusted appropriately to achieve desired drug levels, locally or systemically. Typically, the active agents of the present invention exhibit their effect at a dosage range of from about 0.001 mg/kg to about 250 mg/kg, preferably from about 0.01 mg/kg to about 100 mg/kg, of the active ingredient and more preferably, from about 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, adenosine receptor agonists, local anesthetics, hemostatic agents, potassium channel opener or agonist, AV blockers and therapeutic agents in all of the major areas of clinical medicine. When used with other pharmaceutically active compounds, the conotoxin peptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The κ-PVIIA-related conotoxins and pharmaceutically acceptable salts thereof and their use as organ protecting agents, i.e., organ protectants, as described herein can be used in the treatment of humans or animals, i.e., in veterinary applications. These conotoxins and their use can be utilized for individuals of any age, including pediatric and geriatric patients.

The κ-PVIIA-related conotoxins and pharmaceutically acceptable salts thereof disclosed herein can also be used for the treatment of arrhythmia, urinary incontinence, angina, reperfusion injury, diabetes, retinopathy, neuropathy, nephropathy, peripheral circulation disturbances, acute heart failure, hypertension, cerebral vasospasm accompanying subarachnoid hemorrhage, anxiety disorder, cerebral ischemia, CABG surgery, ischemic heart disease and congestive heart failure. The κ-PVIIA-related conotoxins and pharmaceutically acceptable salts thereof disclosed herein can also be used for open heart surgery, bypass surgery, heart transplant surgery and cardioplegia. Cardioplegia is a technique of myocardial preservation during cardiac surgery usually employing infusion of a cold, potassium laced solution, sometimes fixed with blood, to achieve arrest of the myocardial fibers and reduce their oxygen consumption to nearly nothing. Techniques using warm (body temperature) blood are also used.

Activators of K_{ATP} channels have therapeutic significance for the treatment of asthma, cardiac ischemia and cerebral ischemia, among others.

Asthma: Asthma is a serious and common condition that effects approximately 12 million people in the United States alone. This disorder is particularly serious in children and it has been estimated that the greatest number of asthma patients are those under the age of 18 (National Health Survey, National Center of Health Statistics, 1989). The disease is characterized by chronic inflammation and hyper-responsiveness of the airway which results in periodic attacks of wheezing and difficulty in breathing. An attack occurs when the airway smooth muscle become inflamed and swells as a result of exposure to a trigger substance. In severe cases, the airway may become blocked or obstructed as a result of the smooth muscle contraction. Further exacerbating the problem is the release of large quantities of mucus which also act to block the airway. Chronic asthmatics are most commonly treated prophylactically with inhaled corticosteroids and acutely with inhaled bronchodilators, usually β-2 agonists. However, chronic treatment with inhaled cortico steroids has an associated risk of immune system impairment, hypertension, osteoporosis, adrenal gland malfunction and an increased susceptibility to fungal infections (Rakel, 1997). In addition use of β-2 agonists has been reported in some cases to cause adverse reactions including tremor, tachycardia and palpitations and muscle cramps (Rakel, 1997). Therefore, there is great potential in developing anti-asthmatic agents with fewer side-effects.

K⁺ channel openers have been shown to be effective relaxants of airway smooth muscle reducing hyperactivity induced obstruction of intact airway. In cryopreserved human bronchi (Muller-Schweinitzer and Fozard, 1997) and in the isolated guinea pig tracheal preparation (Lin et al, 1998; Ando et al., 1997; Nielson-Kudsk, 1996; Nagai et al., 1991), K_{ATP} openers produced relaxation whether the muscle was contracted spontaneously or induced by a range of spasmogens. Under these conditions, the K⁺ channel openers are thought to be acting to produce a K⁺ ion efflux and consequent membrane hyperpolarization. As a result, voltage-sensitive Ca²⁺ channels would close and intracellular calcium levels would drop, producing muscular relaxation. The development of new and more specific K_{ATP} openers may offer a novel approach both to the prophylactic and symptomatic treatment of asthma.

K_{ATP} channels are present in many tissue types beyond just the target tissue, therefore their activation may result in unwanted side effects. In particular, as K_{ATP} channels are found in vascular smooth muscle, it is possible that in addition to the beneficial anti-asthmatic properties of K_{ATP} openers there could be an associated drop in blood pressure. It is possible that delivering the compound in inhalant form directly to the airway smooth muscle will allow the concentration of the compound to be reduced significantly thereby minimizing adverse reactions.

Cardiac Ischemia: While numerous subtypes of potassium channels in cardiac tissue have not yet been fully characterized, openers of K_{ATP} channels show great promise as cardioprotective agents. The beneficial vasodilatory effects afforded by K⁺ channel openers in patients with angina pectoris are now well established (Chen et al., 1997; Goldschmidt et al., 1996; Yamabe et al., 1995; Koike et al., 1995). Furthermore, the activation of K_{ATP} channels appears also to be involved in the acute preconditioning of the myocardium following brief ischemic periods, acting to reduce the risk (Pell et al., 1998) and size of the reperfusion infarct (Kouchi et al., 1998).

Direct evidence for the cytoprotective properties of K_{ATP} channels was demonstrated by Jovanovic et al. (1998a). In these studies, the DNA encoding for the Kir6.2/SUR2A (cardiac K_{ATP}) channel were transfected in COS-7 monkey cells and the degree of calcium loading monitored. Untransfected cells were demonstrated to be vulnerable to the increases in intracellular calcium seen following hypoxia/reoxygenation. However, the transfection of the cells with the K_{ATP} channel conferred resistance to the potentially damaging effects of the hypoxia-reoxygenation. Thus, the cardiac K_{ATP} channels are likely to play a significant role in protecting the myocardium against reperfusion injury.

Cerebral Ischemia: Although treatment of cerebral ischemia has advanced significantly over the past 30 years, cerebral ischemia (stroke) still remains the third leading cause of death in the United States. More than 500,000 new stroke/ischemia cases are reported each year. Even though initial mortality is high (38%), there are close to three million survivors of stroke in the United States, and yearly cost for rehabilitation of these patients in the United States is close to $17 billion (Rakel, 1997).

The initial cellular effects occur very rapidly (a matter of minutes) after an ischemic episode, whereas the actual cellular destruction does not occur until several hours or days following the infarction. Initial effects include depolarization due to bioenergetic failure, and inactivation of Na⁺ channels. Voltage-gated calcium channels are activated resulting in a massive rise in intracellular calcium. Further exacerbating the problem is a large transient release of glutamate which itself increases both Na⁺ and Ca²⁺ influx through ionotropic glutamate receptors. Glutamate also binds to metabotropic receptors, which results in activation of the inositol phosphate pathway. This sets off a cascade of intracellular events, including further release of calcium from intracellular stores. It is now well accepted that this initial overload of intracellular calcium ultimately leads to the delayed cytotoxicity that is seen hours or days later.

Recently it has been reported that dopaminergic neurons exposed to a very short hypoxic challenge will hyperpolarize primarily through an opening of K_{ATP} channels (Guatteo et al., 1998). This stimulatory effect was suggested to be a direct result of the increased metabolic demand and the consequent drop in intracellular ATP levels. Furthermore Jovanovic et al. (1998b) recently reported that cells transfected with DNA encoding for Kir6.2/SUR1 (neuronal K_{ATP}) channel showed increased resistance to injury caused through hypoxia-reoxygenation. Therefore, the opening of K_{ATP} channels may serve a vital cytoprotective role during short periods of reduced oxygen in neuronal tissue. Thus, there is great therapeutic potential in developing compounds that not only will act to prevent this calcium influx prophylactically, but will aid in reestablishing the normal resting membrane potential in damaged tissue. Treatment with κ-PVIIA-related conotoxins will act to open K_{ATP} channels, inducing membrane hyperpolarization and indirectly producing closure of the voltage-gated Ca²⁺ channels, thereby preventing or reducing deleterious effects of a massive calcium influx.

In accordance with the present invention, it has been found that intravenous (IV) injection of concentrations of κ-PVIIA, far higher than those required to produce maximal hyperpolarization in tracheal cultures in vitro, had no effect on blood pressure or heart rate in the anesthetized rat.

Our preliminary data indicates that κ-PVIIA induces glibenclamide-sensitive currents in primary cultures of myocytes in a highly potent manner. Furthermore, incubation of primary myocyte cultures in the presence of κ-PVIIA confers protection against hypoxia-induced depolarization. Further data demonstrates that κ-PVIIA reduces the infarct size, thus providing protection to an organ from reperfusion injury.

The present invention also relates to rational drug design for the identification of additional drugs which can be used for the purposes described herein. The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art. Such techniques may include providing atomic coordinates defining a three-dimensional structure of a protein complex formed by said first polypeptide and said second polypeptide, and designing or selecting compounds capable of interfering with the interaction between a first polypeptide and a second polypeptide based on said atomic coordinates.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, and to what extent it is exhibited. Further optimization or modification can then be carried out to arrive at one or more final mimetics for in vivo or clinical testing.

The present invention further relates to the use of a labeled (e.g., radiolabel, fluorophore, chromophore or the like) analog of the κ-PVIIA-related conotoxins described herein as a molecular tool, both in vitro and in vivo, for discovery of small molecules that exert their action at or partially at the same functional site as the native toxin and are capable of eliciting similar functional responses as the native toxin. In one embodiment, the displacement of a labeled κ-PVIIA-related conotoxin from its receptor or other complex by a candidate drug agent is used to identify suitable candidate drugs. In a second embodiment, a biological assay on a test compound to determine the therapeutic activity is conducted and compared to the results obtained from the biological assay of a κ-PVIIA-related conotoxin. In a third embodiment, the binding affinity of a small molecule to the receptor of a κ-PVIIA-related conotoxin is measured and compared to the binding affinity of a κ-PVIIA-related conotoxin to its receptor.

### EXAMPLES

The present invention is described by reference to the following Examples, which are offered by way of illustration and are not intended to limit the invention in any manner. Standard techniques well known in the art or the techniques specifically described below were utilized.

### Example 1

### Experimental Methods

1. Cell Culture Protocol

Primary cultures of rat neonatal cortical cells, ventricular myocytes, tracheal smooth muscle cells and hippocampal cells were prepared. Cortical hemispheres were cleaned of meninges and the hippocampus removed and dissociated separately using 20 U/ml Papain. Cells were dissociated with constant mixing for 45 min at 37° C. Digestion was terminated with fraction V BSA (1.5 mg/ml) and Trypsin inhibitor (1.5 mg/ml) in 10 ml media (DMEM/F12±10% fetal Bovine serum±B27 neuronal supplement; Life Technologies). Cells were gently triturated, to separate cells from surrounding connective tissue. Using a fluid-handling robot (Quadra 96, Tomtec) cells were settled onto Primaria-treated 96 well plates (Becton-Dickinson). Each well was loaded with approximately 25,000 cells. Plates were placed into a humidified 5% CO₂ incubator at 37° C. and kept for at least five days before fluorescence screening. Ventricles were diced into 2 mm square pieces and were digested in the presence of 20 U/ml Papain and trypsin/EDTA 1× (Life technologies). Smooth muscle cells on the surface of the trachea were cultured using the same digestive enzymes. Culturing techniques followed the method above.

2. Fluorimetry Assay

The saline solution used for the fluorimetric assay contained [in mM] 137 NaCl, 5 KCl, 10 HEPES, 25 Glucose, 3 CaCl₂, and 1 MgCl₂.

Di-8-ANEPPs: Voltage-sensitive dye: The effects of the compounds on membrane-potential were examined using the voltage-sensitive dye Di-8-ANEPPs. The Di-8-ANEPPs (2 uM) was dissolved in DMSO (final bath concentration 0.3%) and loaded into the cells in the presence of 10% pluronic acid. The plates were incubated for 40 min and then washed 4 times with the saline solution before starting the experiments. Di-8-ANEPPs crosses over the membrane in the presence of the pluronic acid creating a cytoplasmic pool of dye. Di-8-ANEPPs inserts into the plasma membrane where changes in potential result in molecular rearrangement. During hyperpolarization, the dye interchelates into the outer leaflet of the plasma membrane from the cytoplasmic reservoir of dye. Hyperpolarizations are represented as a positive shift and depolarizations as a negative shift in the fluorescence levels. ANEPPs dyes show a fairly uniform 10% change in fluorescence intensity per 100 mV change in membrane potential and as such, fluorescence changes can be correlated to changes in membrane potential.

PBFI:K⁺ sensitive dye: A lipid-soluble AM ester of the PBFI dye was used to examine the effect of the κ-PVIIA on intracellular potassium levels. The dye was loaded into the cytoplasm with 20% pluronic acid where esterases cleave the dye from the ester effectively trapping the dye within the cell. Increases in intracellular potassium (K⁺i) are reflected as a rise in fluorescence and decreases in K⁺i as a drop in fluorescence. Cells were pre-incubated in 5 uM PBFI for three to four hours prior to screening. As with the Di-8-ANEPPs dye, the plates were rinsed four times with saline prior to beginning the experiments.

Fluo-3-Calcium-sensitive dye: To examine changes in intracellular calcium a lipid-soluble ester of the Fluo-3 dye (2 uM in DMSO. Final bath concentration of DMSO 0.3%) is loaded into the cells in the presence of 20% pluronic acid. The plates are incubated for 35 minutes and washed four times with saline solution before beginning the experiments. Increases and decreases in the concentration of intracellular calcium are reflected as positive and negative changes in the percent fluorescence respectively.

Ethidium homodimer-1: cellular viability dye: The degree of cellular damage produced by a cytotoxic agent was measured using the dye Ethidium homodimer-1(Molecular probes). This dye will not cross intact plasma membranes, but is able to readily enter damaged cells. Upon binding nucleic acids, the dye undergoes a fluorescent enhancement. Thus, the degree of cellular damage can be correlated to the amount of fluorescence. In preparation for the excitotoxicity assay, the cells were rinsed three times and pretreated with the κ-PVIIA or an equal volume of saline. The cells were incubated for 15 minutes and glutamate (5-500 uM) added to the appropriate lanes of the plate. The cells were incubated for a further 30 minutes, and washed thoroughly four times. The Ethidium Dye (4 uM) was loaded into all the wells and a reading was taken immediately. Readings were then taken at hourly intervals.

3. Fluorimetry Protocol

Fluorometric measurements are an averaging of cellular responses from approximately 25,000 cells per well of a 96 well plate. Cultures of cells from the cortex include at least pyramidal neurons, bipolar neurons, interneurons and astrocytes. Changes in membrane potential (Di-8-ANEPPs), cellular damage (Ethidium homodimer-1), intracellular K⁺ (PBFI) and intracellular Ca²⁺(Fluo-3) were used as a measure of the response elicited with κ-PVIIA alone or with κ-PVIIA in the presence of specific receptor/ion channel agonists or antagonists. Concentration-responses were collected with the κ-PVIIA to determine the effective range. In order to minimize well-to-well variability, each well acted as its own control by comparing the degree of fluorescence in pretreatment to that in post-treatment. This normalization process allows comparison of relative responses from plate to plate and culture to culture. Mixed-cell populations in each well were measured with the fluorimeter and individual cell signaling responses were averaged. Statistics, including mean and standard error of the mean, from eight wells allowed for comparison of significant differences between treatments. Results were expressed as percent change in fluorescence. An initial reading of a plate was taken in saline solution. Measurements using the Di-8-ANEPPs, Fluo-3 or PBFI dyes were made at time intervals of 15 seconds, two minutes, five minutes, 10 minutes, 20 minutes and 30 minutes in the presence of the compound. Readings with Ethidium homodimer-1 were made at hourly intervals.

4. Tracheal Smooth Muscle Preparation

Guinea pigs were sacrificed by cervical dislocation and the trachea excised and cleaned of connective tissue. Trachea were cut into four or five sections and opened by cutting through the ring of cartilage opposite the tracheal muscle. Each segment was mounted in a organ bath containing (mM) NaCl 118.2; KCl 4.7; MgSO₄ 1.2; KH₂PO₄ 1.2; Glucose, 11.7; CaCl₂ 1.9 and NaHCO₃ 25.0. The bath was maintained at 37° C. and gassed with 95% O₂ and 5% CO₂. The preparation was maintained under 1 g of tension and equilibrated for 60 minutes before starting the experiment. Contractions were measured isometrically using a force-displacement transducer connected to a Grass polygraph. Following the 60 minutes equilibration period, the trachea were exposed to a submaximal concentration of histamine. This step was repeated until the contractile response to the spasmogen is consistent. The relaxant effects of increasing concentrations of κ-PVIIA was determined in the absence and presence of the histamine.

5. Patch Clamp Recording

Whole-cell patch clamp recordings were made from cortical neurons on coverslips coated with Polyornithine/Poly-D-lysine (5 to 28 days in culture) and from myocytes on uncoated coverslips. Patch pipettes were pulled from thin-wall borosilicate glass and had resistances of 4M to 6M. Currents were recorded with an EPC 9 amplifier (HEKA) and controlled by software (Pulse, (HEKA) run on a Macintosh power PC. Whole-cell currents were low-passed filtered at 10 kHz, digitized through a VR-10b digital data recorder to be stored on videotape at a sampling rate of 94 kHz. The intracellular pipette contained (in mM): 107 KCl, 33 KOH, 10 EGTA, 1 MgCl₂, 1 CaCl₂ and 10 HEPES. The solution was brought to pH 7.2 with NaOH and 0.1-0.5 mM Na₂ATP and 0.1 mM NaADP were added immediately before the experiment. The extracellular solution contained (in mM): 60 KCl, 80 NaCl, 1 MgCl₂, 0.1 CaCl₂ and 10 HEPES. The pH of the external solution was brought to pH 7.4 with NaOH. The high concentration of potassium results in a calculated reversal potential for potassium of −20 mV. As a result, if the holding potential is more negative than −20 mV, opening K⁺ channels will result in an inward flux of K⁺ ions and a downward deflection of the whole cell current. These solutions were chosen as the K_{ATP} channel has weak inward rectifying properties and as such, larger inward currents were anticipated. Experiments that are underway will address the effect of κ-PVIIA in solutions with low potassium levels.

6. Electrophysiology Solutions

Two extracellular solutions were used with different K⁺ ion and Na⁺ ion concentrations. Solution 1 contained 5 mM KCl and has a potassium equilibrium potential (Eₖ) of −84 mV, and solution 2 contained 60 mM and has a corresponding Eₖ of −20 mV. Extracellular solution 1 contained (in mM): 5 KCl, 135 NaCl, 1 MgCl₂, 0.1 CaCl₂ and 10 HEPES. The pH of the external solution was corrected to pH 7.4 with NaOH. Extracellular solution 2 contained (in mM): 60 KCl, 80 NaCl, 1 MgCl₂, 0.1 CaCl₂ and 10 HEPES. The pH of the external solution was corrected to pH 7.4 with NaOH. The intracellular pipette contained (in mM): 107 KCl, 33 KOH, 10 EGTA, 1 MgCl₂, 1 CaCl₂ and 10 HEPES. The solution was brought to pH 7.2 with NaOH and 0.1-0.5 mM Na₂ATP, and 0.1 mM NaADP was added immediately before the experiment.

7. Interpreting the Electrophysiology Results

In the presence of a low concentration of external K⁺ ions (solution 1) and at holding potentials more depolarized than −84 mV, the opening of K⁺ channels will result in an outward flux of K⁺ ions. In the presence of a high concentration of K⁺ (solution 2) the membrane potential would have to be more negative than −20 mV in order to see an outward movement of K⁺ ions. If the actual reversal potentials of the current evoked by κ-PVIIA in two different extracellular solutions are the same as the calculated values, it is highly likely that the κ-PVIIA-induced current is a result of the flux of K⁺ ions. The reversal potential of the current was calculated by holding the cell at the calculated Eₖ and running 500 ms voltage ramps from −100 mV to +80 mV both in the presence and absence of increasing concentrations of κ-PVIIA. The average of four control ramps was subtracted from the average of four ramps evoked in the presence of κ-PVIIA. The resultant trace was the actual current induced by the presence of the compound. This was fitted with a polynomial function and the reversal potential calculated.

8. Time-Lapse Confocal Ca²⁺ Imaging

Cortical cell cultures were loaded with the fluorescent Ca²⁺ indicator Fluo3-AM (Molecular Probes, Eugene Oreg.; 2 mM final concentration with 0.1% Pluronic acid) 40 minutes prior to imaging experiments. Coverslips containing cells were mounted in a laminar flow perfusion chamber (Cornell-Bell design; Warner Instruments, Hamden, Conn.) and rinsed in saline (137 mM NaCl, 5 mM KCl, 3 mM CaCl₂, 1 mM MgCl₂, 10 mM HEPES, and 20 mM Sorbitol, pH 7.3) for at least five minutes to remove excess Fluo-3AM. Time-lapse images were collected on a Nikon PCM200 (Melville, N.Y.) confocal scanning laser microscope equipped with a Zeiss Axiovert135 inverted microscope (Carl Zeiss, Inc., Thornwood, N.Y.) and downloaded with no frame averaging every 1.8 seconds to an optical memory disk recorder (Panasonic TQ3031F, Secaucus N.J.) (see methods further described in Kim et al., 1994). Image analysis was performed on a standardized 5×5 pixel area of cytoplasm in every astrocyte in the field to prevent bias in data analysis. Time course plots of intensity measurements (% change in fluorescence) were obtained using programs written by H. Sontheimer (Birmingham, Ala.) and plotted using Origin (MicroCal Northampton, Mass.). Routine analysis consisted of time course plots for up to 200 cells per field with at least five trials, thus yielding data analysis often from thousands of cells per experiment.

### Example 2

### Exposure to κ-PVIIA Produces a Dose-Dependent Decrease in Intracellular K⁺

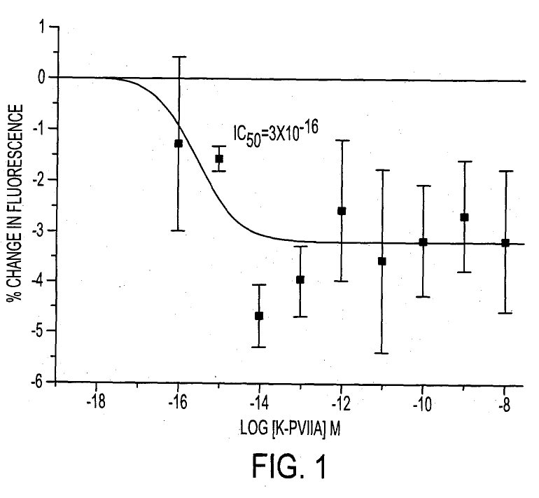

κ-PVIIA was originally isolated from the purple cone snail (Conus purpurascens) and was found to block the Drosophila H4 shaker K⁺ channel (Shon et al, 1998). In the same study no effects of the peptide were noted in oocytes expressing the mammalian shaker-like voltage-sensitive K⁺ channels Kv1.1 and Kv1.3. The potential of the peptide to block other voltage-gated K⁺ channels present in primary cultures of cortex was tested in this study. A 96-well fluorimetry assay was used to look for changes in potassium levels under depolarized conditions where voltage-gated potassium channels (Kv) would be activated. The cells were preloaded with the potassium indicator dye PBFI. If the compound acted to block Kv channels in a depolarized environment, there would be a resultant increase in intracellular K⁺. The results, however, suggested that at concentrations up to 100 nM, there was a reduction in the intracellular K⁺ concentration in untreated resting preparations (FIG. 1), as well as those preparations depolarized with 10-100 uM Aconitine. While the changes in fluorescence in the PBFI dye evoked with κ-PVIIA are small, it is important to stress that they are significant and repeatable.

### Example 3

### Exposure to κ-PVIIA Produces Dose-Dependent Hyperpolarization

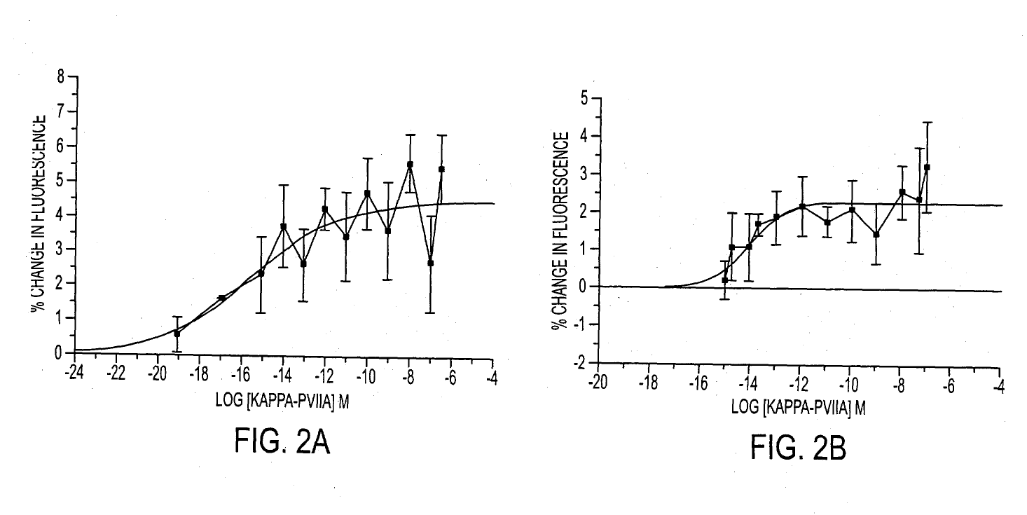

The fluorimetry experiments were repeated in the presence of the voltage-sensitive dye Di-8-ANEPPs, and the drop in intracellular K⁺ levels was seen to be accompanied by a significant hyperpolarization of the preparation (represented by a positive shift in the fluorescence, FIGS. 2A-2B). κ-PVIIA is extremely potent in this assay, showing EC₅₀s of 8×10⁻¹⁶ M in cortex, 9×10⁻¹⁶ M in myocyte cultures and 9×10⁻¹⁸ M in primary cultures of tracheal myocytes.

### Example 4

### The κ-PVIIA-Induced Hyperpolarization is Blocked by Exposure to K_{ATP} Antagonists

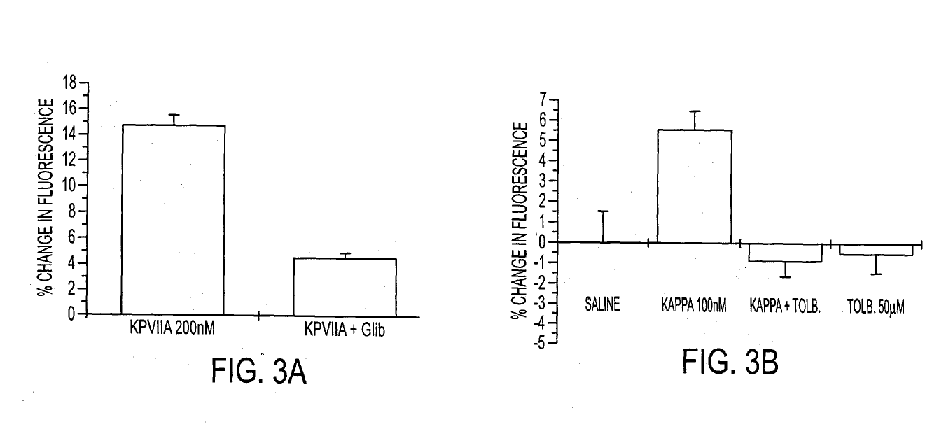

In order to determine the involvement of different K⁺ channel subtypes in the κ-PVIIA-induced hyperpolarization, effects of five well-documented K⁺ channel antagonists (4-aminopyridine (4-AP), Iberiotoxin (IBTX), Apamin, Tolbutamide and Glibenclamide) were tested. In cortical preparations, applications of 4-AP, IBTX and Apamin were without any detectable effect on the hyperpolarization seen with 100 nM κ-PVIIA. However, both Tolbutamide (1-10 uM) and Glibenclamide (10 nM), antagonists of the K_{ATP} channel, produced significant reductions in the κ-PVIIA induced hyperpolarization (FIG. 3B). Glibenclamide also produced significant reductions in the κ-PVIIA-induced hyperpolarization in cultures of myocytes (FIG. 3A).

### Example 5

### κ-PVIIA Induces Tolbutamide or Glibenclamide-Sensitive Currents

The sensitivity of the response to K_{ATP} antagonists was confirmed using the whole-cell patch clamp technique. In these experiments, the extracellular potassium concentration was increased to 60 mM and the solutions were calculated such that the reversal potential for potassium (Eₖ) would be −20 mV. Thus, the opening of K⁺ channels when the membrane potential is more negative than −20 mV will result in an influx of K⁺ ions. In both primary cultures of cortex and cardiac myocytes, the superfusion of 100 nM κ-PVIIA induced an inward flux of positive ions that reversed close to −20 mV, indicating the involvement of K⁺ ions. With a holding potential of −80 mV, the currents evoked by κ-PVIIA were significantly larger in the myocyte preparation (87.7±5.9 pA, n=8) compared to the cortical preparation (26.2±6.2 pA, n=4). Even when the currents are corrected for cell capacitance, responses produced by the myocytes were greater than those seen in the cortical preparation (4.6±0.4 pA/pf and 2.4±0.7 pA/pf, respectively).

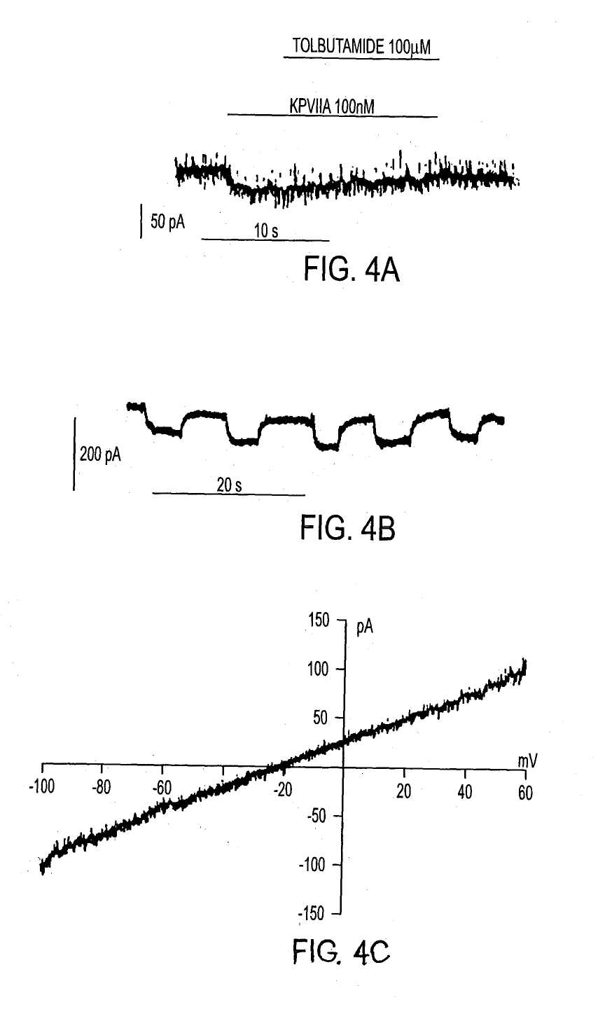

In both cases, the currents were sensitive either to the K_{ATP} antagonists tolbutamide (100 uM) or glibenclamide (10 nM) (FIGS. 4A and B). The reversal potential of the κ-PVIIA evoked current was determined using a voltage ramp from −100 to +60 mV and fitting the results with a fourth-order polynomial fit (FIG. 4C). The experimentally determined Eₖ (−23 mV) was close to the calculated Eₖ of −20 mV for these high potassium solutions, indicating the involvement of K⁺ channels.

### Example 6

### κ-PVIIA Produces a Slowly Developing Reduction in Intracellular Calcium

The effects of κ-PVIIA on intracellular calcium levels were determined using a 96-well fluorimetry assay plate and loading the cells with the Ca²⁺ indicator dye Fluo-3. In primary cultures of cortical neurons, κ-PVIIA produced a significant reduction in intracellular calcium. Little effect was noticeable with 1 nM κ-PVIIA at 15 seconds (−2.15±0.95%, two trials) but over time, the drop in calcium concentration became more profound (30 min, −8.8±3.9%).

### Example 7

### κ-PVIIA Protects Against Hypoxia-Induced Depolarization

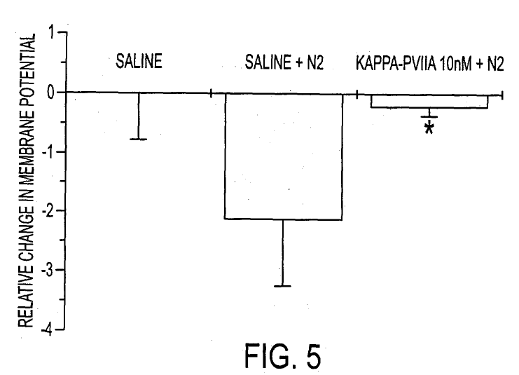

The depolarizing effects of N₂-induced hypoxia have been monitored in cardiac ventricular myocytes using the voltage sensitive dye Di-8-ANEPPs in a 96 well fluorimetry assay plate. Solutions were depleted of oxygen by constant bubbling with N₂ gas and were compared to results with control untreated saline. Under these conditions, hypoxia produced significant depolarization of the preparation (reflected as a drop in fluorescence), and incubating the preparation with 10 nM κ-PVIIA prevented any hypoxia-induced changes in membrane potential (FIG. 5).

### Example 8

### κ-PVIIA Protects Against Glutamate-Induced Excitotoxicity

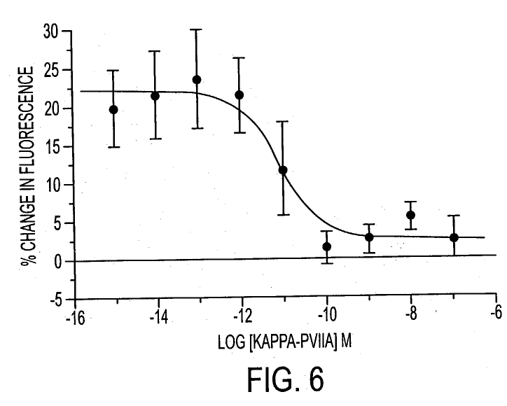

The protective effect of κ-PVIIA against glutamate-induced excitotoxicity was tested, using the 96-well fluorimetry assay and the Ethidium homodimer-1 dead cell dye. Five lanes of the 96-well plate were pre-exposed to 100 pM κ-PVIIA, and another five to control saline. Glutamate was then applied for 30 minutes, at which time the entire plate was washed thoroughly to remove all κ-PVIIA and glutamate. Ethidium dye was loaded, an initial reading taken and the amount of delayed cytotoxicity monitored for six hours. Increases in fluorescence represent increased cell destruction. As can be seen from FIG. 6, pre-incubating the cortical cells in κ-PVIIA resulted in very effective protection against the delayed (6 hrs) cytotoxic effects of 100 uM glutamate. This protection was blocked by 100 uM tolbutamide (K_{ATP} antagonist).

### Example 9

### Cytotoxicity of κ-PVIIA

Incubation of primary cortical cultures with 200 nM κ-PVIIA for 20 minutes induced no detectable protease activity (three trials). In comparison, a 20 minutes incubation with 5% Triton produced an ˜14% increase in fluorescence, as detected by the Enzchek protease-sensitive dye.

### Example 10

### Evaluating Protective Ability of κ-PVIIA in in vitro Model of Hypoxia

A combination of the 96-well fluorimetric assay, electrophysiology, and confocal microscopy are used to assess the ability of κ-PVIIA to protect against the acute effects of transiently depleting oxygen in primary cultures. A multi-chamber saline reservoir has been constructed that allows the lower half of delivery plate to be filled with saline that is bubbled with N₂. Individual chambers allow the effects of decreasing oxygen to be monitored in the presence and absence of different concentrations of the κ-PVIIA. An initial screen in primary cultures of ventricular myocytes, using the potentiometric dye Di-8-ANEPPs, shows a strong protective effect of the κ-PVIIA against hypoxia induced depolarization. Similar effects are seen in the cortex and trachea. When the calcium-sensitive dye fluo-3 is used to observe changes in intracellular calcium levels induced by the hypoxic challenge, it is seen that κ-PVIIA is able to provide protection against hypoxia in all three tissue preparations. A similar result is obtained using the current-clamp mode of the whole cell patch clamp technique to monitor changes in membrane potential induced by hypoxia electrophysiology. This technique is very sensitive and allows the examination of the effect of κ-PVIIA on single tracheal, neuronal or myocyte cells.

### Example 11

### Evaluating Protective Ability of κ-PVIIA in in vitro Model of Excitotoxicity

Preliminary fluorimetric experiments monitoring the degree of delayed cellular death produced following a challenge to a high concentration of glutamate have been carried out in primary cultures of cortex. The results indicate that the presence of the κ-PVIIA effectively reduces the degree of glutamate-induced excitotoxicity in a dose-dependant manner. Using the current-clamp mode of the whole-cell patch clamp technique, correlation of the fluorimetry results to actual changes in the membrane potential is examined. It is seen that the presence of the κ-PVIIA prevents the initial glutamate-induced depolarization, thereby conferring protection against the glutamate-induced calcium influx.

### Example 12

### Effect of κ-PVIIA on Infarct Size

Initially, the effect of κ-PVIIA on infarct size in isolated rabbit hearts was analyzed. In this model, an infarct is induced in isolated hearts by a 30 min occlusion of the coronary artery followed by 2 hours of reperfusion. It was found that a 10 min perfusion with 10 nM and 100 nM κ-PVIIA reduced the infarct size. It was also found that a 10 min perfusion with 1 nM κ-PVIIA had no effect on infarct size. In view of these results, an in vivo model was used for further analysis.

In this study, the ability of κ-PVIIA to salvage myocardium when given just prior to reperfusion was tested. This study was performed in accordance with The Guide for the Care and Use of Laboratory Animals (National Academy Press, Washington, D.C., 1996).

New Zealand White rabbits of either sex weighing 1.6-2.7 kg were anesthetized with pentobarbital (30 mg/kg iv), intubated through a tracheotomy, and ventilated with 100% oxygen via a positive pressure respirator. The ventilation rate and tidal volume were adjusted to maintain arterial blood gases in the physiological range. Body temperature was maintained at 38-39° C. A catheter was inserted into the left carotid artery for monitoring blood pressure. Another catheter was inserted into the right jugular vein for drug infusion. A left thoracotomy was performed in the fourth intercostal space, and the pericardium was opened to expose the heart. A 2-0 silk suture on a curved taper needle was passed through the myocardium around a prominent branch of the left coronary artery. The ends of the suture were passed through a small piece of soft vinyl tubing to form a snare. Ischemia was induced by pulling the snare and then fixing it by clamping the tube with a small hemostat. Ischemia was confirmed by appearance of cyanosis. All animals received an ischemic insult of 30 min (the index ischemia) to create an infarct. Reperfusion was achieved by releasing the snare and was confirmed by visible hyperemia on the ventricular surface.

After 3 h of reperfusion, the rabbit was given an overdose of pentobarbital and the heart was quickly removed from the chest, mounted on a Langendorff apparatus, and perfused with saline to wash out blood. Then the coronary artery was reoccluded, and 5 ml of 0.1% Fluorescent microspheres (1-10 μm diameter, Duke Scientific Corp, Palo Alto, Calif.) were infused into the perfusate to demarcate the risk zone as the area of tissue without fluorescence. The heart was weighed, frozen, and cut into 2.5-rom-thick slices. The slices were incubated in 1% triphenyltetrazolium chloride (TTC) in sodium phosphate buffer at 37° C. for 20 min. The slices were immersed in 10% formalin to enhance the contrast between stained (viable) and unstained (necrotic) tissue and then squeezed between glass plates spaced exactly 2 mm apart. The myocardium at risk was identified by illuminating the slices with ultraviolet light. The infarcted and risk zone areas were traced on a clear acetate sheet by an investigator blinded to the treatment and quantified with digital planimetry. The areas were converted into volumes by multiplying the areas by slice thickness. Infarct size is expressed as a percentage of the risk zone.

The protocols were as follow. Group I served as a control and after 20 min stabilization, underwent the 30 min period of occlusion followed by 3 hr Reperfusion. Group 2 experienced 5 min of preconditioning (PC) and served as a positive control for a known protective intervention. Group 3 received 10 μg/kg κ-PVIIA as an intravenous bolus 5 min prior to reperfusion. Group 4 received 100 μg/kg κ-PVIIA 5 min prior to reperfusion. Two other groups were included. Because a new investigator was used in this project, he did a small group with 10 μg/kg κ-PVIIA given as a bolus 10 min prior to the index ischemia to see if he could duplicate the data of the previous investigator. A final group was studied where 100 μg/kg κ-PVIIA was given 10 minutes after reperfusion. This would test whether the drug exerted its protection at reperfusion.

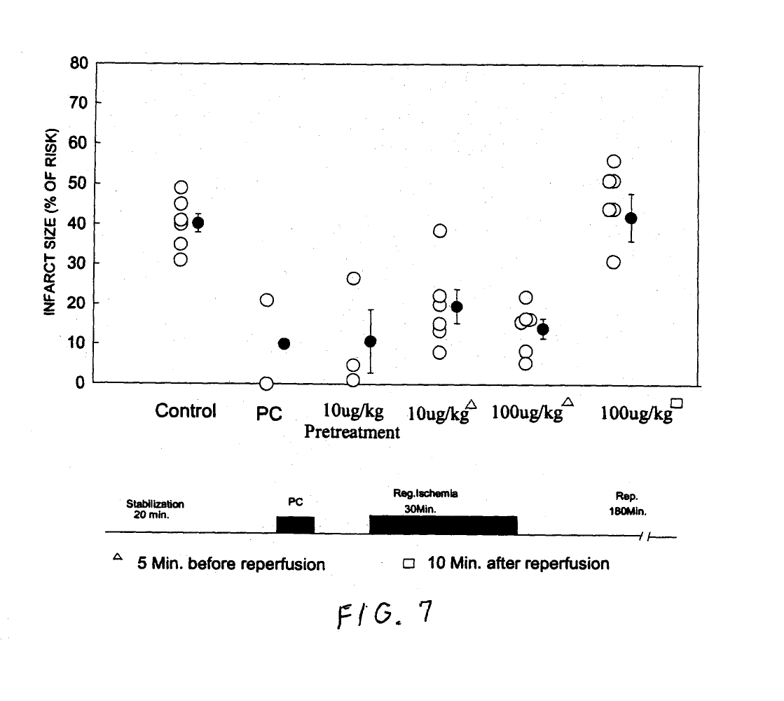

FIG. 7 shows the resulting infarct sizes when expressed as a % of the risk zone. Note that PC caused a dramatic reduction in infarct size as has been our past experience. Pretreatment with κ-PVIIA also caused a robust protective effect almost as potent as PC. Both 10 and 100 μg/kg doses given just prior to reperfusion were also equally as protective (p<0.003 vs. Control, ANOVA). When the drug was started 10 min after reperfusion protection was lost (p=NS vs. Control).

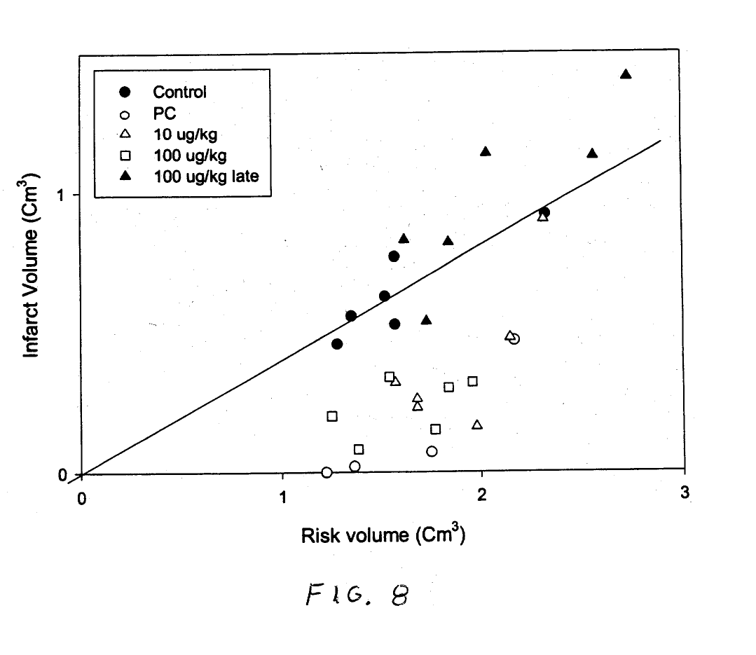

FIG. 8 shows infarct sizes plotted against the region at risk. Experience has shown that infarct size is not exactly proportional to the risk zone size in untreated rabbits but usually has a zero infarct size with a risk size of about 0.35 cm (Xu et al., 2000). Although the non-zero intercept is not apparent in this particular control group it can be shown to occur when larger groups are analyzed. The effect of this relationship is that risk zone can independently influence the infarct size when infarct size is normalized as a percentage of the risk zone. In effect, groups that appear to be protected may not be protected but simply have smaller risk sizes. We test for this artifact by plotting infarct against the risk zone size. The line shows the regression for the control hearts (black circles). Protection is denoted by a shift of the relationship downward. Notice that all hearts in all protected groups fall below the control regression indicating true protection.

κ-PVIIA was found to be without any hemodynamic effect at either dose. All animals tend to have a fall in blood pressure in the latter stage of reperfusion due to the stress of the prolonged surgical procedure.

These results reveal that κ-PVIIA is just as protective when administered just prior to reperfusion as it is when given as a pretreatment. Many drugs can limit infarct size when given as a pretreatment such as sodium hydrogen exchange inhibitors (cariporide) and the preconditioning mimetics which include adenosine and other Gi-coupled receptor agonists and the mitochondrial K_{ATP} openers such as diazoxide. Unfortunately, none of these agents are protective if given once ischemia has started. Pretreatment is seldom an option in the clinical setting, however, since patients do not present until a coronary thrombosis has already occurred. What is needed is a drug that will salvage myocardium when it is administered after ischemia has started. κ-PVIIA seems to fulfill that requirement. We would envision κ-PVIIA being used in acute myocardial infarction patients as an adjunct to thrombolysis and direct angioplasty.

There are very few drugs that have been identified that can protect at reperfusion. In the 1980's it was proposed that free radical scavengers could limit infarct size if they were in the plasma during reperfusion. Unfortunately, virtually all of those reports have proven to be irreproducible and it seems unlikely that this class of agents is effective. We have been involved with a drug currently under development by Aventis, AMP579 (Xu et al., 2001a; Xu et al., 2000). AMP579 is an adenosine A1/A2 receptor agonist and has similar potency to κ-PVIIA. Pharmacology reveals that the A2a receptor is involved in AMP579's protection as blockers of this subtype abolish the protection but interesting A2a agonists or adenosine itself cannot duplicate AMP579's effect (Xu et al., 2001b).

Another class of drugs which appear to protect at reperfusion is the growth factor receptor agonists. Urocortin is the best studied of this class (Latchman, 2001) although TGF-β1 has also been reported to protect (Baxter et al., 2001). The common feature of all of these drugs that protect at reperfusion is that the ERK (Extracellular Receptor Kinase, AKA: p42/p44 MAP kinase) inhibitor, PD 98059, blocks the protection suggesting that ERK activation may be involved (Baxter et al., 2001). Why ERK activation would be protective is unknown nor has it been proven that PD 98059 blocks protection by blocking ERK as opposed to some non-specific effect.

### Example 13

### Effect of κ-PVIIA in Canine Model of AMI

To confirm activity in a second species the cardioprotective effect of κ-PVIIA was also assessed in an open-chest barbital anesthetized canine model of AMI. For a general reference to this model, see Mizumura et al. (1995).

For these studies anaesthetized dogs (˜15 kg) were subjected to a 60 min occlusion of the left anterior descending coronary artery (LAD) followed by a 3 hour reperfusion period. All dogs were instrumented for the measurement of hemodynamics. Radioactive microspheres were used to measure regional blood flow. Following the reperfusion period the hearts were removed and stained with TTZ as for the rabbit model to determine the degree of infarct damage. Four groups of dogs were treated with either vehicle or κ-PVIIA at 30, 100 or 300 μg/kg given as an IV bolus 5 min prior to the release of the occluding snare (55 min following occlusion).

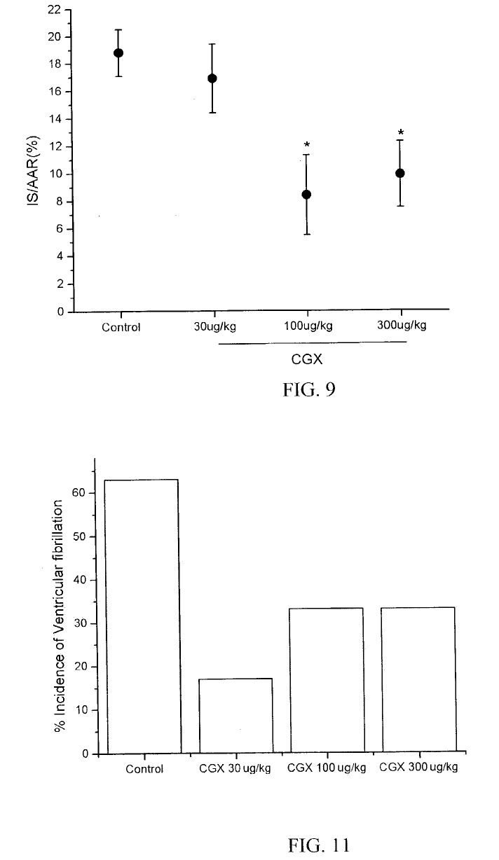

As can be seen from FIG. 9 IV administration of κ-PVIIA at doses of 100 μg/kg and 300 μg/kg showed significant protection reducing the infarct size by approximately 60%. No significant effect was seen at the lower dose of 30 ug/kg.

Thus this study confirmed cardioprotective activity in a second species although the lowest effective dose was slightly higher in the dogs as compared to the rabbits (100 μg/kg vs. 10 μg/kg).

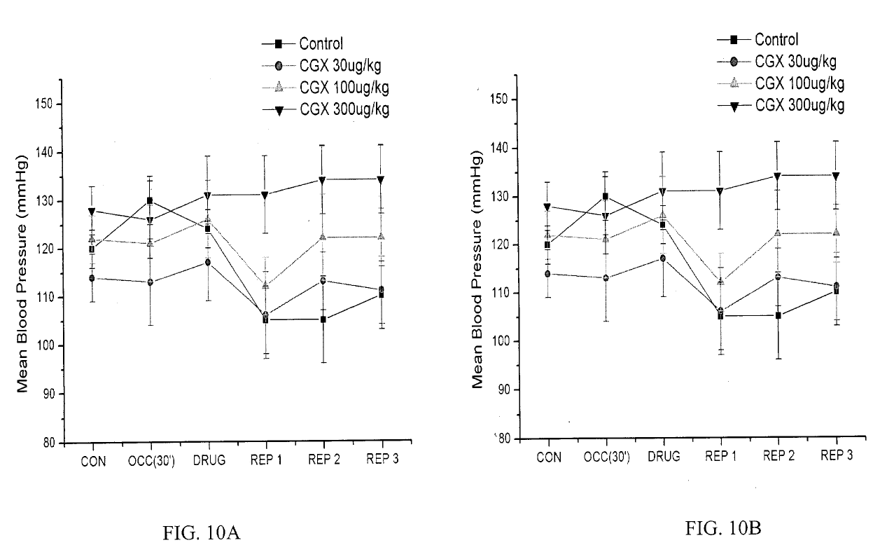

As with the rabbit studies no reduction in blood pressure (FIG. 10A) or heart rate (FIG. 10B) was noted at any dose of κ-PVIIA. In fact at the highest dose of 300 ug/kg κ-PVIIA actually prevented the drop in blood pressure that is normally seen upon reperfusion in this canine model.

In this canine model it is not unusual for the dogs to experience ventricular fibrillation immediately following release of the occluding snare. This normally requires electrical shocking to return the heart back to normal sinus rhythm. A noteworthy finding was that the incidence of ventricular fibrillation was less following administration of κ-PVIIA at all of the doses studied when compared to controls (FIG. 11). While this finding was not statistically significant, probably due to the small sample size (n=6), it is certainly indicative of an anti-arrhythmic effect.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, described embodiments are illustrative and should not be construed as restrictive.

### BIBLIOGRAPHY

Ando, T. et al. (1997). Clin. Exp. Allergy. 27:706-713.

Barnay, G. et al. (2000). J. Med. Chem.

Barnes, P. J. (1992). Eur. Respir. J. 5:1126-1136.

Baxter, G. F. et al. (2001). J Cardiovas Pharmacol 38:930-939.

Bitan, G. et al. (1997). J. Peptide Res. 49:421-426.

Chen, J. W. et al. (1997) Am. J. Cardiol. 80: 32-38.

Craik, D. J. et al. (2001). Toxicon 39:43-60.

Ettinger, L. J. et al. (1978). Cancer 41:1270-1273.

Garcia, E. et al. (1999). J. Gen. Physiol. 114:141-157.

Goldschmidt, M. et al. (1996). J. Clin. Pharmacol. 36:559-572.

Grover, G. J. (1996). Potassium Channel Modulators, 1996, Vol 1, No. 2, pp.7-16.

Guatteo, E. et al. (1998). J. Neurophysiol. 79:1239-1245.

Hammerland et al. (1992). Eur. J. Pharmacol. 226:239-244.

Hubry, V. et al. (1994). Reactive Polymers 22:231-241.

Jovanovic, A. et al. (1998a). Circulation 98:1548-1555.

Jovanovic, A. et al. (1998b). Lab. Invest. 78:1101-1107.

Jung, Y. S. et al. (1998). Japan J. Pharmacol. 76(1):65-73.

Kim et al. (1994). GLIA 11:173-184.

Koike, A. et al. (1995). Am J. Cardiol. 76:449-452.

Kouchi, I. et al. (1998). Am. J. Physiol. 274(4/2); H1106-H1112.

Latchman, D. S. (2001). Trends Cardiovasc Med 11:167-169.

Lauritzen I. et al. (1997). J. Neurochem. 69(4); 1570-1579.

Lin, C. et al. (1998). Pharmacology 57(6); 314-322.

Lauer, M. S. & Hatton, J. (1993). Annals Pharmcotherapy 27:912-921.

Mizumura, T. et al. (1995). Circulation 92:1236-1245.

Morley, J. (1994). Trends Pharmacol. Sci. 15(12); 463-468.

Muller-Shweinitzer, E. and Fozard, J. R.(1997). Br. J. Pharmacol. 120(7):1241-1248.

Nagai, H. et al. (1991). Japan J. Pharmacol. 56(1):13-21.

Neilson-Kudsk, J. E. (1996). Dan. Med. Bull. 43(5): 429-447.

Noma, A. (1983). Nature 304:147-148.

Ornstein, et al. (1993). Biorganic Medicinal Chemistry Letters 3:43-48.

Pell, T. J. et al. (1998). Am. J. Physiol. 275(5 pt 2):H542-547.

Rakel, R. E. (1997). Conn's current therapy.

Remington's Pharmaceutical Sciences, 18th Ed. (1990, Mack Publishing Co., Easton, Pa.).

Reshef A. et al. (1998). Neurosci. Lett. 250(2):111-114.

Sambrook, J. et al., (1989). Molecular Cloning: A Laboratory Manual, 2nd Ed., Cold Spring Harbor Laboratory, Cold Spring Harbor, N.Y.

Shon, K.-J. et al. (1998). J. Biol. Chem. 273:33-38.

Terlau, H. et al. (1996). Nature 381:148-151.

Terlau, H. et al. (1999). J. Gen. Physiol. 114:125-140.

Van de Steen, P. et al. (1998). Critical Rev. in Biochem. and Mol. Biol. 33:151-208.

Xu, Z. et al. (2000). J Moll Cell Cardiol 32:2339-2347.

Xu, Z. et al. (2001a). J. Cardiovasc Pharmacol 38:474-481.

Xu, Z. et al. (2001b). Circulation 104:II-149 (abstract).

Wind, T. et al. (1997). Brain Res. 751(2):295-299.

Yamabe H. et al. (1995). Cardiovasc. Drugs Ther. 9(6):755-761.

Zimm, S. et al. (1984). Cancer Res. 44:1698-1701.

U.S. Pat. No. 5,331,001.

U.S. Pat. No. 5,534,615.

U.S. Pat. No. 5,364,769.

U.S. Pat. No. 5,545,723.

U.S. Pat. No. 5,550,050.

U.S. Pat. No. 5,844,077.

U.S. Pat. No. 5,672,682.

PCT Published Application WO 92/19195.

PCT Published Application WO 94/25503.

PCT Published Application WO 95/01203.

PCT Published Application WO 95/05452.

PCT Published Application WO 96/02286.

PCT Published Application WO 96/02646.

PCT Published Application WO 96/11698.

PCT Published Application WO 96/40871.

PCT Published Application WO 96/40959.

PCT Published Application WO 97/12635.

PCT Published Application WO 00/23092.

What is claimed is:

1. A method for arresting, protecting and/or preserving an organ of a subject mammal which comprises administering to a subject mammal or organ in need thereof an effective amount of a compound that binds to the κ-PVIIA-binding site.

2. The method of claim 1, wherein said compound is a κ-PVIIA-related conotoxin.

3. The method of claim 2, wherein said κ-PVIIA-related conotoxin is selected from the group consisting of κ-PVIIA, E6.2, P6.1, P6.3, congeners thereof, analogs thereof and derivatives thereof.

4. The method of claim 1, wherein the organ is either intact in the body of the subject or isolated.

5. The method of claim 1, wherein the organ is selected from the group consisting of a circulatory organ, respiratory organ, urinary organ, digestive organ, reproductive organ, endocrine organ, neurological organ or somatic cell.

6. The method of claim 5, wherein the circulatory organ is a heart.

7. The method of claim 6, wherein the heart is arrested, protected or preserved during open heart surgery, cardioplegia, angioplasty, valve surgery, transplantation, angina or cardiovascular disease so as to reduce heart damage before, during or following cardiovascular intervention or to protect those portions of the heart that have been starved of normal flow of blood, nutrients and/or oxygen.

8. The method of claim 2, wherein the organ is selected from the group consisting of a circulatory organ, respiratory organ, urinary organ, digestive organ, reproductive organ, endocrine organ, neurological organ or somatic cell.

9. The method of claim 8, wherein the circulatory organ is a heart.

10. The method of claim 9, wherein the heart is arrested, protected or preserved during open heart surgery, cardioplegia, angioplasty, valve surgery, transplantation, angina or cardiovascular disease so as to reduce heart damage before, during or following cardiovascular intervention or to protect those portions of the heart that have been starved of normal flow of blood, nutrients and/or oxygen.

11. The method of claim 3, wherein the organ is selected from the group consisting of a circulatory organ, respiratory organ, urinary organ, digestive organ, reproductive organ, endocrine organ, neurological organ or somatic cell.

12. The method of claim 11, wherein the circulatory organ is a heart.

13. The method of claim 12, wherein the heart is arrested, protected or preserved during open heart surgery, cardioplegia, angioplasty, valve surgery, transplantation, angina or cardiovascular disease so as to reduce heart damage before, during or following cardiovascular intervention or to protect those portions of the heart that have been starved of normal flow of blood, nutrients and/or oxygen.

14. The method of claim 1, wherein an adenosine receptor agonist is also administered to said subject mammal or said organ.

15. The method of claim 14, wherein the adenosine receptor agonist is selected from the group consisting of CPA, NECA, CGS-21680, AB-MECA, AMP579, 9APNEA, CHA, ENBA, R-PIA, DPMA, CGS-21680, ATL146e, CCPA, CI-IB-MECA, IB-MECA.

16. The method of claim 2, wherein an adenosine receptor agonist is also administered to said subject mammal or said organ.

17. The method of claim 16, wherein the adenosine receptor agonist is selected from the group consisting of CPA, NECA, CGS-21680, AB-MECA, AMP579, 9APNEA, CHA, ENBA, R-PIA, DPMA, CGS-21680, ATL146e, CCPA, CI-IB-MECA, IB-MECA.

18. The method of claim 1, wherein a local anesthetic is also administered to said subject mammal or said organ.

19. The method of claim 18, wherein the local anesthetic is selected from the group consisting of mexilitine, diphenylhydantoin, prilocaine, procaine, mipivicaine, bupivicaine, lidocaine and class 1B anti-arrhythmic agents.

20. The method of claim 19, wherein the class 1B anti-arrhythmic agent is lignocaine.

21. The method of claim 2, wherein a local anesthetic is also administered to said subject mammal or said organ.

22. The method of claim 21, wherein the local anesthetic is selected from the group consisting of mexilitine, diphenylhydantoin, prilocaine, procaine, mipivicaine, bupivicaine, lidocaine and class 1B anti-arrhythmic agents.

23. The method of claim 22, wherein the class 1B anti-arrhythmic agent is lignocaine.

24. The method of claim 14, wherein a local anesthetic is also administered to said subject mammal or said organ.

25. The method of claim 16, wherein a local anesthetic is also administered to said subject mammal or said organ.

26. The method of claim 1, wherein a potassium channel opener or agonist is also administered to said subject mammal or said organ.

27. The method of claim 26, wherein the potassium channel opener or agonist is selected from the group consisting of cromakalin, pinacidil, nicorandil, NS-1619, diazoxide, and minoxidil.

28. The method of claim 2, wherein a potassium channel opener or agonist is also administered to said subject mammal or said organ.

29. The method of claim 28, wherein the potassium channel opener or agonist is selected from the group consisting of cromakalin, pinacidil, nicorandil, NS-1619, diazoxide, and minoxidil.

30. The method of claim 14, wherein a potassium channel opener or agonist is also administered to said subject mammal or said organ.

31. The method of claim 16, wherein a potassium channel opener or agonist is also administered to said subject mammal or said organ.

32. The method of claim 18, wherein a potassium channel opener or agonist is also administered to said subject mammal or said organ.

33. The method of claim 21, wherein a potassium channel opener or agonist is also administered to said subject mammal or said organ.

34. The method of claim 24 wherein a potassium channel opener or agonist is also administered to said subject mammal or said organ.

35. The method of claim 25, wherein a potassium channel opener or agonist is also administered to said subject mammal or said organ.

36. The method of claim 1, wherein a hemostatic agent is also administered to said subject mammal or said organ.

37. The method of claim 36, wherein the hemostatic agent is selected from the group consisting of a clot buster agent, a thrombolytic agent, an anti-coagulant agent, an anti-platelet aggregation agent and combination thereof.

38. The method of claim 37, wherein the clot buster agent is selected from the group consisting of streptokinase, urokinase and ACTIVASE.

39. The method of claim 37, wherein the thrombolytic agent is selected from the group consisting of streptokinase, urokinase, alteplase, reteplase and tenecteplase.

40. The method of claim 37, wherein the anti-coagulant agent is selected from the group consisting of heparin, enoxaparin and dalteparin.

41. The method of claim 37, wherein the anti-platelet aggregation agent is selected from the group consisting of aspirin, clopidogrel, abciximab, eptifibatide and tirofiban.

42. The method of claim 2, wherein a hemostatic agent is also administered to said subject mammal or said organ.

43. The method of claim 42, wherein the hemostatic agent is selected from the group consisting of a clot buster agent, a thrombolytic agent, an anti-coagulant agent, an anti-platelet aggregation agent and combination thereof.

44. The method of claim 43, wherein the clot buster agent is selected from the group consisting of streptokinase, urokinase and ACTIVASE.

45. The method of claim 43, wherein the thrombolytic agent is selected from the group consisting of streptokinase, urokinase, alteplase, reteplase and tenecteplase.

46. The method of claim 43, wherein the anti-coagulant agent is selected from the group consisting of heparin, enoxaparin and dalteparin.

47. The method of claim 43, wherein the anti-platelet aggregation agent is selected from the group consisting of aspirin, clopidogrel, abciximab, eptifibatide and tirofiban.

48. The method of claim 14, wherein a hemostatic agent is also administered to said subject mammal or said organ.

49. The method of claim 16, wherein a hemostatic agent is also administered to said subject mammal or said organ.

50. The method of claim 18, wherein a hemostatic agent is also administered to said subject mammal or said organ.

51. The method of claim 21, wherein a hemostatic agent is also administered to said subject mammal or said organ.

52. The method of claim 24, wherein a hemostatic agent is also administered to said subject mammal or said organ.

53. The method of claim 25, wherein a hemostatic agent is also administered to said subject mammal or said organ.

54. The method of claim 26, wherein a hemostatic agent is also administered to said subject mammal or said organ.

55. The method of claim 28, wherein a hemostatic agent is also administered to said subject mammal or said organ.

56. The method of claim 30, wherein a hemostatic agent is also administered to said subject mammal or said organ.

57. The method of claim 31, wherein a hemostatic agent is also administered to said subject mammal or said organ.

58. The method of claim 32, wherein a hemostatic agent is also administered to said subject mammal or said organ.

59. The method of claim 33, wherein a hemostatic agent is also administered to said subject mammal or said organ.

60. The method of claim 34, wherein a hemostatic agent is also administered to said subject mammal or said organ.

61. The method of claim 35, wherein a hemostatic agent is also administered to said subject mammal or said organ.

62. The method of claim 1, wherein an AV blocker is also administered to said subject mammal or said organ.

63. The method of claim 62, wherein the AV blocker is verapamil.

64. The method of claim 2, wherein an AV blocker is also administered to said subject mammal or said organ.

65. The method of claim 64, wherein the AV blocker is verapamil.

66. The method of claim 14, wherein an AV blocker is also administered to said subject mammal or said organ.

67. The method of claim 16, wherein an AV blocker is also administered to said subject mammal or said organ.

68. The method of claim 18, wherein an AV blocker is also administered to said subject mammal or said organ.

69. The method of claim 21, wherein an AV blocker is also administered to said subject mammal or said organ.

70. The method of claim 24, wherein an AV blocker is also administered to said subject mammal or said organ.

71. The method of claim 25, wherein an AV blocker is also administered to said subject mammal or said organ.

72. The method of claim 26, wherein an AV blocker is also administered to said subject mammal or said organ.

73. The method of claim 28, wherein an AV blocker is also administered to said subject mammal or said organ.

74. The method of claim 30, wherein an AV blocker is also administered to said subject mammal or said organ.

75. The method of claim 31, wherein an AV blocker is also administered to said subject mammal or said organ.

76. The method of claim 32, wherein an AV blocker is also administered to said subject mammal or said organ.

77. The method of claim 33, wherein an AV blocker is also administered to said subject mammal or said organ.

78. The method of claim 34, wherein an AV blocker is also administered to said subject mammal or said organ.

79. The method of claim 35, wherein an AV blocker is also administered to said subject mammal or said organ.

80. The method of claim 36, wherein an AV blocker is also administered to said subject mammal or said organ.

81. The method of claim 42, wherein an AV blocker is also administered to said subject mammal or said organ.

82. The method of claim 48, wherein an AV blocker is also administered to said subject mammal or said organ.

83. The method of claim 49, wherein an AV blocker is also administered to said subject mammal or said organ.

84. The method of claim 50, wherein an AV blocker is also administered to said subject mammal or said organ.

85. The method of claim 51, wherein an AV blocker is also administered to said subject mammal or said organ.

86. The method of claim 52, wherein an AV blocker is also administered to said subject mammal or said organ.

87. The method of claim 53, wherein an AV blocker is also administered to said subject mammal or said organ.

88. The method of claim 54, wherein an AV blocker is also administered to said subject mammal or said organ.

89. The method of claim 55, wherein an AV blocker is also administered to said subject mammal or said organ.

90. The method of claim 56, wherein an AV blocker is also administered to said subject mammal or said organ.

91. The method of claim 57, wherein an AV blocker is also administered to said subject mammal or said organ.

92. The method of claim 58, wherein an AV blocker is also administered to said subject mammal or said organ.

93. The method of claim 59, wherein an AV blocker is also administered to said subject mammal or said organ.

94. The method of claim 60, wherein an AV blocker is also administered to said subject mammal or said organ.

95. The method of claim 61, wherein an AV blocker is also administered to said subject mammal or said organ.

96. The method of claim 1, wherein each agent or combination of agents is administered by a route selected from the group consisting of oral, rectal, intracerebralventricular, intrathecal, epidural, intravenous, intramuscular, subcutaneous, intranasal, transdermal, transmucosal, sublingual, by irrigation, by release pump or by infusion.

97. The method of claim 96, wherein the route is intravenous and each agent or combination of agents is administered either continuously or intermittantly.

98. The method of claim 97, wherein each agent or combination of agents is mixed with donor blood prior to delivery to the subject, provided that the donor blood is compatible with that of the subject.

99. A method for identifying drug candidates for use as organ arresting, protecting or preserving agents which comprises screening a drug candidate for its action at, or partially at, the same functional site as a κ-PVIIA-related conotoxin and its capability of elucidating a similar functional response as said conotoxin.

100. The method of claim 99, wherein the displacement of a labeled κ-PVIIA-related conotoxin from its receptor or other complex by a candidate drug agent is used to identify suitable candidate drugs.

101. The method of claim 99, wherein a biological assay on a test compound to determine the therapeutic activity is conducted and compared to the results obtained from the biological assay of a κ-PVIIA-related conotoxin.

102. The method of claim 99, wherein the binding affinity of a small molecule to the receptor of a κ-PVIIA-related conotoxin is measured and compared to the binding affinity of a κ-PVIIA-related conotoxin to its receptor.
